SEQUENCE LISTING (3 sequences)

SEQ ID NO: 1 - protein, 570 aa, Unknown
  Yeast
MSLLIDSVPTVAYKDQKPGTSGLRKKTKVFMDEPHYTENFIQATMQSIPNGSEGTTLVVGGDGRFYNDVIMNKIAAVGAANGVRKLVIGQGGLLSTPAASHIIRTYEEKCTGGGIILTASHNPGGPENDLGIKYNLPNGGPAPESVTNAIWEASKKLTHYKIIKNFPKLNLNKLGKNQKYGPLLVDIIDPAKAYVQFLKEIFDFDLIKSFLAKQRKDKGWKLLFDSLNGITGPYGKAIFVDEFGLPAEEVLQNWHPLPDFGGLHPDPNLTYARTLVDRVDREKIAFGAASDGDGDRNMIYGYGPAFVSPGDSVAIIAEYAPEIPYFAKQGIYGLARSFPTSSAIDRVAAKKGLRCYEVPTGWKFFCALFDAKKLSICGEESFGTGSNHIREKDGLWAIIAWLNILAIYHRRNPEKEASIKTIQDEFWNEYGRTFFTRYDYEHIECEQAEKVVALLSEFVSRPNVCGSHFPADESLTVIDCGDFSYRDLDGSISENQGLFVKFSNGTKFVLRLSGTGSSGATIRLYVEKYTDKKENYGQTADVFLKPVINSIVKFLRFKEILGTDEPTVRT

SEQ ID NO: 2 - protein, 562 aa, Unknown
  Bovine
MVKIVTVKTKAYQDQKPGTSGLRKRVKVFQSSSNYAENFIQSIISTVEPAQRQEATLVVGGDGRFYMKEAIQLIVRIAAANGIGRLVIGQNGILSTPAVSCIIRKIKAIGGIILTASHNPGGPNGDFGIKFNISNGGPAPEAITDKIFQISKTIEEYAICPDLHVDLGVLGKQQFDLENKFKPFTVEIVDSVEAYATMLRNIFDFNALKELLSGPNRLKIRIDAMHGVVGPYVKKILCEELGAPANSAVNCVPLEDFGGHHPDPNLTYAADLVETMKTGEHDFGAAFDGDGDRNMILGKHGFFVNPSDSVAVIAANIFSIPYFQQTGVRGFARSMPTSGALDRVANATKIALYETPTGWKFFGNLMDASKLSLCGEESFGTGSDHIREKDGLWAVLAWLSILATRKQSVEDILKDHWQKYGRNFFTRYDYEEVEAEGANKMMKELEALISDRSFVGKQFPVGDKVYTVEKIDNFEYSDPVDGSISRNQGLRLLFADGSRIIFRLSGTGSAGATIRLYIDSYEKDLAKIYQDPQVMLAPLISIALKVSQLQEKTGRTAPTVIT

SEQ ID NO: 3 - protein, 562 aa, Unknown
  Equine
MVKIVTVKTQAYPDQKPGTSGLRKRVKVFQSSAHYAENFIQSILSTVEPAQRQEATLVVGGDGRFYMKEAIQLIVRIAAANGIGRLVIGQNGILSTPAVSCIIRKIKAIGGIILTASHNPGGPNGDFGIKFNISNGGPAPEAITDKIFQISKTIEEYAICPDLKVDLGVLGKQQFDLENKFKPFTVEIVDSVEAYATMLRNIFDFNALKELLSGPNRLKIRIDAMHGVVGPYVKKILCEELGAPANSAVNCVPLEDFGGHHPDPNLTYAADLVETMKTGEHDFGAAFDGDGDRNMILGKHGFFVNPSDSVAVIAANIFSIPYFQQTGVRGFARSMPTSGALDRVANATKIALYETPTGWKFFGNLMDASKLSLCGEESFGTGSDHIREKDGLWAVLAWLSILATRKQSVEDILKDHWQKYGRNFFTRYDYEEVAAEGANKMMKDLEALITDRSFVGKQFSEGDKVYTVEKIDNFEYSDPVDGSISRNQGLRLIFADGSRIIFRLSGTGSAGATIRLYIDSYEKDLAKIYQDPQVMLAPLISIALKVSKLQERTGRTAPTVIT

(19) United States
(12) Patent Application Publication
(10) Pub. No.: US 2023/0407244 A1
(43) Pub. Date: Dec. 21, 2023

(54) MICROBIAL GROWTH-STIMULATING PROTEIN AND METHODS OF USING SAME

(71) Applicant: Paradigm Diagnostics, Inc., Chanhassen, MN (US)

(72) Inventor: Alan D Olstein, Mendota Heights, MN (US)

(73) Assignee: Paradigm Diagnostics, Inc., Chanhassen, MN (US)

(21) Appl. No.: 18/250,637

(22) PCT Filed: Oct. 26, 2021
(86) PCT No.: PCT/US2021/056588

§ 371 Date: Apr. 26, 2023

Related U.S. Application Data
(60) Provisional application No. 63/192,293, filed on May 24, 2021.
(60) Provisional application No. 63/105,929, filed on Oct. 27, 2020.

(51) Int. Cl. C12N 1/38; C12N 1/20

(52) U.S. Cl. CPC C12N 1/38; C12N 1/20

(57) ABSTRACT

Microbial growth media and methods of growing a microorganism. The microbial growth media may include a growth-stimulating amount of phosphoglucomutase in combination with one or more of a carbon and nitrogen source, a fermentable sugar, and an inorganic salt. The methods of growing a microorganism may include culturing a sample suspected of containing the microorganism in a microbial growth medium containing a growth-stimulating amount of phosphoglucomutase. Microbial growth-stimulating protein compositions and uses of same are also provided.

### BACKGROUND

The frequency of food recalls linked to the presence of pathogenic E. coli, such as Shiga-toxin producing E. coli (STEC), continue to increase in the U.S. and many other industrialized countries. Likewise, Salmonella is a common environmental and gastrointestinal tract-associated pathogen that can contaminate food. Methods for screening food products for these pathogens have primarily been prescribed by the USDA Microbiology Laboratory Guide (U.S. Department of Agriculture, Food Safety Inspection Service. 2014. Microbiology Laboratory Guidebook. Method no. 5B.05) and FDA Bacteriological Assay Manual (U.S. Food and Drug Administration. Peter Feng and Karen Jinneman B A M: Diarrheagenic Escherichia coli. 2017). Considerable innovation has been applied to improving these processes through the introduction of lateral flow immunoassay methods, PCR, and whole genome sequencing. To accomplish all of the above-mentioned methods, the sample is usually enriched to permit the target population to reach the limit of detection for the intended method as well as to ensure that the analyte measured is from viable microorganisms.

In this regard, work has been focused on optimizing enrichment media and methods to increase efficiency of target pathogen recovery from food and environmental samples. One particular enrichment medium, Salmonella Indicator Broth (PDX-SIB), employs the use of efflux pump inhibitors for enrichment of Salmonella and pathogenic E. coli (U.S. Pat. Nos. 9,518,283; 9,029,118; Olstein et al. 2013 (Olstein A, Griffith L, Feirtag J, Pearson N. Paradigm Diagnostics Salmonella Indicator Broth (PDX-SIB) for detection of Salmonella on selected environmental surfaces. J AOAC Int. 2013 March-April; 96(2):404-12)). Studies demonstrate that the PDX-SIB medium can be used to simultaneously enrich for Salmonella sp. and STEC contamination (Eggers et al. 2018).

Factors that enhance the growth of microbes such as STEC and Salmonella are needed.

### SUMMARY OF THE INVENTION

The invention is directed to microbial growth-stimulating protein, compositions containing same, and methods of using same.

A first aspect of the invention is directed to microbial growth media. One microbial growth medium of the invention comprises a growth-stimulating amount of phosphoglucomutase and a component comprising a carbon and nitrogen source, a fermentable sugar, an inorganic salt, and any combination thereof. Another microbial growth medium of the invention comprises a microbial growth-stimulating protein composition of the invention and a component comprising a carbon and nitrogen source, a fermentable sugar, an inorganic salt, and any combination thereof. Exemplary combinations of the carbon and nitrogen source, the fermentable sugar, and the inorganic salt include the carbon and nitrogen source and the fermentable sugar; the fermentable sugar and the inorganic salt; the carbon and nitrogen source and the inorganic salt; and the carbon and nitrogen source, the fermentable sugar, and the inorganic salt.

Another aspect of the invention is directed to methods of growing a microorganism. One method comprises culturing a sample suspected of containing the microorganism in a microbial growth medium of the invention. Another method comprises culturing a sample suspected of containing the microorganism in a microbial growth medium comprising a growth-stimulating amount of phosphoglucomutase.

Another aspect of the invention is directed to methods of preparing a microbial growth-stimulating protein composition. One method comprises incubating an extraction liquid comprising water and a surfactant with an animal meat for a time sufficient to extract microbial growth-stimulating protein from the animal meat into the extraction liquid to thereby generate a conditioned composition. The method further comprises separating the microbial growth-stimulating protein from at least a portion of at least one other component of the conditioned composition. The method further comprises sterilizing the microbial growth-stimulating protein. The culmination of these steps results in a microbial growth-stimulating protein composition of the invention.

Another aspect of the invention is directed to a microbial growth-stimulating protein compositions made by the methods of the invention.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Flow chart of experimental approach.

FIG. 2. Growth curves for STEC O157:H7 (A), STEC-O111 (B), and STEC-O121 (C) in SSS media (Cntrl; square) and in SSS media containing 10% v/v F-1 preparation from ammonium sulfate precipitation of ground beef extract (F-1-STEC; circle) measured at 3, 5 and 7 h post inoculation.

FIG. 3. Growth curves for Salmonella Newport (A), Salmonella Heidelberg (B), and Salmonella Tennessee (C) in modified SSS medium (Cntrl; square) and in modified SSS medium containing 10% v/v F-1 fraction from ammonium sulfate precipitation of ground beef extract (F-1-STEC; circle) measured at 3, 5 and 7 h post inoculation.

FIG. 4. Effect of Phoshoglucomutase (PGM) containing F-1 fraction on the growth of E. coli O157:H7 wheat samples. Medium (modified Buffered Peptone Water with pyruvate; mBPWp) supplemented with PGM (10% F-1; circle) compared to control using mBPWp (square) was measured over 7 h of incubation at 42° C.

FIG. 5. Platings of turkey enrichments in PDX-STEC medium supplemented with a 20-60% ammonium sulfate cut of ground beef extract. (A) shows the 2.0 and 10 CFU/g sample at 6.5 hr enrichment versus the control. (B) shows the 0.5, 2.0 and 10 CFU/g samples at 10 hr enrichment versus the control.

FIG. 6. Platings of turkey enrichments in PDX-STEC medium without supplementation with the 20-60% ammonium sulfate cut of ground beef extract. The figure shows the 2.0 and 10 CFU/g samples at 6.5 hr enrichment versus the control.

### DETAILED DESCRIPTION OF THE INVENTION

Aspects of the invention are directed to microbial growth-stimulating proteins and/or microbial growth-stimulating protein compositions. The microbial growth-stimulating protein compositions of the invention comprise a microbial growth-stimulating protein. A preferred microbial growth-stimulating protein of the invention is phosphoglucomutase. As outlined in the following examples, phosphoglucomutase is shown to be capable of being extracted from various sources, including animal meat and yeast, and having growth-stimulating activity. Thus, in preferred versions of the invention, the microbial growth-stimulating protein and/or the microbial growth-stimulating protein compositions of the invention comprise phosphoglucomutase.

“Phosphoglucomutase” as used herein refers to any polypeptide having phosphoglucomutase activity (EC 5.4.2.2). Exemplary phosphoglucomutases include the bovine, equine, and yeast phosphoglucomutases discussed in the following examples. An exemplary yeast phosphoglucomutase has the following amino acid sequence:

(SEQ ID NO: 1)
MSLLIDSVPTVAYKDQKPGTSGLRKKTKVFMDEPHYTENF
IQATMQSIPNGSEGTTLVVGGDGRFYNDVIMNKIAAVGAA
NGVRKLVIGQGGLLSTPAASHIIRTYEEKCTGGGIILTAS
HNPGGPENDLGIKYNLPNGGPAPESVTNAIWEASKKLTHY
KIIKNFPKLNLNKLGKNQKYGPLLVDIIDPAKAYVQFLKE
IFDFDLIKSFLAKQRKDKGWKLLFDSLNGITGPYGKAIFV
DEFGLPAEEVLQNWHPLPDFGGLHPDPNLTYARTLVDRVD
REKIAFGAASDGDGDRNMIYGYGPAFVSPGDSVAIIAEYA
PEIPYFAKQGIYGLARSFPTSSAIDRVAAKKGLRCYEVPT
GWKFFCALFDAKKLSICGEESFGTGSNHIREKDGLWAIIA
WLNILAIYHRRNPEKEASIKTIQDEFWNEYGRTFFTRYDY
EHIECEQAEKVVALLSEFVSRPNVCGSHFPADESLTVIDC
GDFSYRDLDGSISENQGLFVKFSNGTKFVLRLSGTGSSGA
TIRLYVEKYTDKKENYGQTADVFLKPVINSIVKFLRFKEI
LGTDEPTVRT

An exemplary bovine phosphoglucomutase has the following amino acid sequence:

(SEQ ID NO: 2)
MVKIVTVKTKAYQDQKPGTSGLRKRVKVFQSSSNYAENFI
QSIISTVEPAQRQEATLVVGGDGRFYMKEAIQLIVRIAAA
NGIGRLVIGQNGILSTPAVSCIIRKIKAIGGIILTASHNP
GGPNGDFGIKFNISNGGPAPEAITDKIFQISKTIEEYAIC
PDLHVDLGVLGKQQFDLENKFKPFTVEIVDSVEAYATMLR
NIFDFNALKELLSGPNRLKIRIDAMHGVVGPYVKKILCEE
LGAPANSAVNCVPLEDFGGHHPDPNLTYAADLVETMKTGE
HDFGAAFDGDGDRNMILGKHGFFVNPSDSVAVIAANIFSI
PYFQQTGVRGFARSMPTSGALDRVANATKIALYETPTGWK
FFGNLMDASKLSLCGEESFGTGSDHIREKDGLWAVLAWLS
ILATRKQSVEDILKDHWQKYGRNFFTRYDYEEVEAEGANK
MMKELEALISDRSFVGKQFPVGDKVYTVEKIDNFEYSDPV
DGSISRNQGLRLLFADGSRIIFRLSGTGSAGATIRLYIDS
YEKDLAKIYQDPQVMLAPLISIALKVSQLQEKTGRTAPTV
IT

An exemplary equine phosphoglucomutase has the following amino acid sequence:

(SEQ ID NO: 3)
MVKIVTVKTQAYPDQKPGTSGLRKRVKVFQSSAHYAENFI
QSILSTVEPAQRQEATLVVGGDGRFYMKEAIQLIVRIAAA
NGIGRLVIGQNGILSTPAVSCIIRKIKAIGGIILTASHNP
GGPNGDFGIKFNISNGGPAPEAITDKIFQISKTIEEYAIC
PDLKVDLGVLGKQQFDLENKFKPFTVEIVDSVEAYATMLR
NIFDFNALKELLSGPNRLKIRIDAMHGVVGPYVKKILCEE
LGAPANSAVNCVPLEDFGGHHPDPNLTYAADLVETMKTGE
HDFGAAFDGDGDRNMILGKHGFFVNPSDSVAVIAANIFSI
PYFQQTGVRGFARSMPTSGALDRVANATKIALYETPTGWK
FFGNLMDASKLSLCGEESFGTGSDHIREKDGLWAVLAWLS
ILATRKQSVEDILKDHWQKYGRNFFTRYDYEEVAAEGANK
MMKDLEALITDRSFVGKQFSEGDKVYTVEKIDNFEYSDPV
DGSISRNQGLRLIFADGSRIIFRLSGTGSAGATIRLYIDS
YEKDLAKIYQDPQVMLAPLISIALKVSKLQERTGRTAPTV
IT

The following examples show that fragments of full-length, native phosphoglucomutase proteins have growth-stimulating activity. Phosphoglucomutases of the invention accordingly comprise active fragments of full-length, native phosphoglucomutase proteins. The fragments preferably comprise at least 15% by mass, at least 20% by mass, at least 25% by mass, at least 30% by mass, at least 35% by mass, at least 40% by mass, at least 45% by mass, at least 50% by mass, at least 55% by mass, at least 60% by mass, at least 65% by mass, at least 70% by mass, at least 75% by mass, at least 80% by mass, at least 85% by mass, at least 90% by mass, at least 95% by mass, at least 99% or 100% by mass of a full, native phosphoglucomutase protein, wherein the mass is determined by SDS-PAGE. The fragments preferably comprise a number of amino acid residues of at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% of the number of amino acid residues of a full, native phosphoglucomutase protein.

Polypeptides having the same amino acid sequence as a phosphoglucomutase but lacking phosphoglucomutase activity (EC 5.4.2.2) due to unfolding, denaturing, post-translational modification, heat-inactivation, or other modifications are not considered herein to constitute a phosphoglucomutase.

The invention provides microbial growth media comprising microbial growth-stimulating protein of the invention. The microbial growth media can comprise any base media capable of supporting growth of at least one microbe combined with the microbial growth-stimulating protein and/or microbial growth-stimulating protein composition of the invention. The base media can include any microbial growth media known in the art or developed in the future. Exemplary media to which the microbial growth-stimulating protein of the invention can be added include any of those of U.S. Pat. Nos. 9,518,283 and 9,029,118, which are incorporated herein by reference.

The microbial growth-stimulating protein of the invention is preferably included in the enhanced microbial growth medium of the invention in an amount effective to confer enhanced microbial growth stimulating activity to the microbial growth medium with respect to a medium lacking the microbial growth-stimulating protein of the invention but otherwise identical to the microbial growth medium (e.g., the base medium). The enhanced microbial growth stimulating activity is preferably for a bacterium, such as a Gram-negative bacterium, such as E. coli or Salmonella. The E. coli is preferably STEC. In some versions, the microbial growth-stimulating protein is phosphoglucomutase. In some versions, the phosphoglucomutase comprises a phosphoglucomutase other than bovine phosphoglucomutase. In some versions, the phosphoglucomutase comprises yeast phosphoglucomutase. “Yeast phosphoglucomutase” refers to phosphoglucomutase natively found in yeast. “Bovine phosphoglucomutase” refers to phosphoglucomutase natively found in bovines. “Equine phosphoglucomutase” refers to phosphoglucomutase natively found in equines.

The invention provides methods of partially purifying phosphoglucomutase from yeast in a way that preserves phosphoglucomutase activity. See the following examples. Accordingly, in some versions of the invention, the phosphoglucomutase is provided in a microbial growth-stimulating protein composition in the form of a partially purified yeast protein preparation. “Partially purified yeast protein preparation” refers to a protein preparation obtained from yeast in which at least one component in the original intact yeast has been removed from the phosphoglucomutase originally present in the original intact yeast. In some versions, the partially purified yeast protein preparation comprises at least one yeast protein other than phosphoglucomutase. “Yeast protein” in this context refers to a protein natively found in yeast. The yeast from which the phosphoglucomutase in the partially purified yeast protein preparation can be genetically modified to enhance production of the native phosphoglucomutase and/or to express or overexpress a heterologous phosphoglucomutase. Methods of genetically modifying yeast to enhance production of native proteins or to express or overexpress heterologous proteins are well known in the art. Accordingly, in some versions, the partially purified yeast protein preparation comprises a heterologous phosphoglucomutase. “Heterologous phosphoglucomutase” in this context refers to a phosphoglucomutase not natively found in the yeast from which the partially purified yeast protein preparation is derived.

In some versions, the phosphoglucomutase comprises an amino acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO:1. The term “identical” (or “identity”), in the context of two polynucleotide or polypeptide sequences, means that the residues in the two sequences are the same when aligned for maximum correspondence, as measured using a sequence comparison or analysis algorithm (see, for example, U.S. Pat. No. 10,844,410, which is incorporated herein by reference in its entirety). For example, if when properly aligned, the corresponding segments of two sequences have identical residues at 5 positions out of 10, it is said that the two sequences have a 50% identity. Most bioinformatic programs report percent identity over aligned sequence regions, which are typically not the entire molecules. If an alignment is long enough and contains enough identical residues, an expectation value can be calculated, which indicates that the level of identity in the alignment is unlikely to occur by random chance. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

In some versions, the microbial growth media of the invention is sterile. The microbial growth medium can be made to be sterile using any sterilization method, including those explicitly described herein.

The microbial growth media of the invention may include a carbon and nitrogen source. Exemplary carbon and nitrogen sources include protein hydrolysates and/or extracts. Suitable carbon and nitrogen sources include peptone, neopeptone, tryptone beef extract paste, desiccated powder of beef heart, desiccated powder of beef liver, brain heart infusion, digests of casein, and yeast extract. Examples include the following products from BD (Franklin Lakes NJ): ACIDICASE™ Peptone (hydrochloric acid hydrolysis of casein; Cat. No. 211843); Beef Extract Paste (Cat. No. 212610); Beef Heart for Infusion (Desiccated powder of beef heart, Cat. No. 213210); BIOSATE™ Peptone (Cat No. 211862); BIOSATE™ Peptone (Cat. No. 294312); Brain Heart Infusion (Cat. No. 237300); Casamino acids (acid hydrolyzed casein; Cat. Nos. 223050, 223020, 223120, 223030, 223110, 228820, 228830, and 228830); Casein Digest (Enzymatic digest of casein for molecular genetics, Cat. No. 211610); Casitone (Pancreatic digest of casein; Cat. Nos. 225930 and 225910); Gelatin (Cat. Nos. 214340 and 214320); GELYSATE™ Peptone (Pancreatic digest of gelatin; Cat. No. 211870); Liver (Desiccated powder of beef liver; Cat. No. 213320); Neopeptone (Enzymatic digest of protein; Cat. Nos. 211680 and 211681); Peptone (An enzymatic digest of protein; Cat Nos. 211830, 211677, 254820, 211820); PHYTONE™ Peptone (An enzymatic digest of soybean meal, Non-animal origin; Cat. Nos. 211906 and 298147); PHYTONE™ Peptone UF (Ultra-filtered enzymatic digest of soybean meal, designed specifically for cell culture applications, non-animal origin; Cat. Nos. 210931 and 210936); Polypeptone Peptone (Pancreatic digest of casein and peptic digest of animal tissue combined in equal parts; Cat. Nos. 211910 and 297108); Proteose Peptone (Enzymatic digest of protein, high in proteoses; Cat. Nos. 212010, 253310, and 211684); Proteose Peptone No. 2 (Enzymatic digest of protein; Cat. Nos. 212120 and 212110); Proteose Peptone No. 3 (Enzymatic digest of protein; Cat. Nos. 211693, 211692, 212220, and 212230) Proteose Peptone No. 4 (Enzymatic digest of protein; Cat. No. 211715); Select Soytone (Enzymatic digest of soybean meal, non-animal origin; Cat. Nos. 212489 and 212488); Soytone, BACTO™ (Enzymatic hydrolysate of soybean meal; Cat. Nos. 243620 and 243610); TC Yeastolate (Water soluble portion of autolyzed yeast, source of Vitamin B complex, tested for tissue culture; Cat. Nos. 255772 and 255771); TRYPTICASE™ Peptone (Enzymatic digest of casein; Cat. No. 211921); TRYPTICASE™ Peptone (Enzymatic digest of casein; Cat. Nos. 211922 and 211923); Tryptone (Enzymatic digest of casein; Cat. Nos. 211705, 211701, and 211699); Tryptone, BITEK™ (Enzymatic digest of casein; Cat. No. 251420); Tryptose (Enzymatic hydrolysate of protein; Cat. Nos. 211709 and 211713); Yeast Extract (Water-soluble extract of autolyzed yeast cells suitable for use in culture media; Cat. Nos. 212720, 211931, 211929, 212710, 212730, 211930, and 212750); Yeast Extract, LD (Water-soluble extract of autolyzed yeast cells that has been agglomerated to minimize dusting; Cat. Nos. 210933 and 210941); Yeast Extract, UF (Water-soluble extract of autolyzed yeast cells, ultra-filtration enhances solubility and lowers the endotoxin, suitable for use in cell culture and microbial fermentation; Cat. Nos. 210934 and 210929); and equivalents thereof. Peptone or tryptone supplemented with beef or yeast extract are preferred carbon and nitrogen sources. Exemplary concentration ranges of the carbon and nitrogen source include concentrations from about 1 g/L to about 300 g/L, such as from about 2 g/L to about 150 or from about 10 g/L to about 30 g/L.

The microbial growth media of the invention may include an inorganic salt. Suitable inorganic salts include calcium salts, copper salts, iron salts, selenium salts, potassium salts, magnesium salts, sodium salts, ammonium salts, nickel salts, tin salts, and zinc salts, among others. Suitable examples of such salts include CaCl₂), CuSO₄, FeSO₄, H₂SeO₃, KCl, KI, KH₂PO₄, MgCl₂, MgCO₃, MgSO₄, MnSO₄, Na₂HPO₄, Na₂SiO₃, NaCl, NaH₂PO₄, NaHCO₃, NH₄VO₃, (NH₄)₆Mo₇O₂₄, NiCl₂, SnCl₂, ZnSO₄, and hydrates thereof Magnesium, potassium, calcium, iron and/or zinc salts are preferred. Magnesium salts, such as magnesium chloride (MgCl₂), magnesium carbonate (MgCO₃), magnesium sulfate (MgSO₄), and hydrates thereof are particularly preferred. A particularly suitable magnesium salt is magnesium chloride. The inorganic salt is preferably included at a concentration sufficient to create high osmotic pressure in the medium. Exemplary concentration ranges of the inorganic salt include concentrations from about 0.01 g/L to about 50 g/L, such as from about 0.1 g/L to about 40 g/L, from about 0.5 g/L to about 35 g/L, or from about 1 g/L to about 15 g/L.

The microbial growth media of the invention may include a pH indicator. The pH indicator is preferably sensitive to acidification. The pH indicator is preferably an indicator that transitions color in a range of about pH 7 to about pH 5. Examples of suitable pH indicators are bromocresol purple, phenol red, and neutral red. Bromocresol purple is preferred. The pH indicator may be included in any concentration suitable for detecting the pH change. Exemplary concentration ranges of the pH indicator include concentrations from about 0.004 g/L to about 0.25 g/L, such as about 0.02 g/L to about 0.05 g/L.

The microbial growth media of the invention may include a fermentable sugar. Examples of suitable sugars include adonitol, arabinose, arabitol, ascorbic acid, 5-bromo-4-chloro-3-indolyl-β-D-galactopyranoside (X-gal), chitin, D-cellubiose, 2-deoxy-D-ribose, dulcitol, (S)-(+)-erythrulose, fructose, fucose, galactose, glucose, isopropyl 3-D-1-thiogalactopyranoside (IPTG), inositol, lactose, lactulose, lyxose, maltitol, maltose, maltotriose, mannitol, mannose, melezitose, melibiose, microcrystalline cellulose, palatinose, pentaerythritol, raffinose, rhamnose, ribose, sorbitol, sorbose, starch, sucrose, trehalose, xylitol, xylose, and hydrates thereof. For selection of Salmonella spp., it is preferred to use a sugar that can be efficiently metabolized by Salmonella spp. but not by other bacteria. 2-Deoxy-D-ribose, xylose, mannitol, dulcitol, sorbitol, L-rhamnose and D-arabitol are suitable for this purpose. 2-Deoxy-D-ribose is particularly preferred because of its relative selectivity toward Salmonella enterica. It was previously thought that, with the exception of a few Citrobacter species, most non-Salmonella species within Enterobacteriacae are incapable of fermenting 2-deoxy-D-ribose (Tourneux, L. et al. Genetic and Biochemical Characterization of Salmonella enterica Serovar Typhi Deoxyribokinase. J. Bact. 182(4):869-873. 2000; and Christensen, M. et al. Regulation of Expression of the 2-Deoxy-D-Ribose Utilization Regulon, deoQKPX, from Salmonella enterica Serovar Typhimurium. J. Bact. 185(20):6042-6050. 2003). However, more recent publications have provided evidence that some Klebsiella and Enterobacter species can also ferment 2-Deoxy-D-ribose (Hansen et al. Recommended Test Panel for Differentiation of Klebsiella Species on the Basis of a Trilateral Interlaboratory Evaluation of 18 Biochemical Tests. J. Clin. Microbiol. 42(8):3665-3669. 2004). Exemplary concentrations of the fermentable sugar include concentrations from about 0.5 g/L to about 120 g/L, such as from about 1.0 g/L to about 60 g/L or from about 5.0 to about 12.0 g/L.

The microbial growth media of the invention may include one or more visual indicators. “Visual indicators” as used herein denote components that provide a visual indication of the presence of one or more types of microorganisms. Exemplary visual indicators include pH indicators. The microbial growth media of the invention preferably include one or more visual indicators that indicate the presence of Salmonella; E. coli, such as Shiga toxin-producing E. coli; or both Salmonella and E. coli. Exemplary indicators include the combination of a pH indicator such as bromocresol purple and 2-deoxy-D-ribose for indicating the presence of Salmonella, the combination of 5-bromo-4-chloro-3-indolyl-β-D-galactopyranoside (X-gal) and isopropyl β-D-1-thiogalactopyranoside (IPTG) for indicating the presence of E. coli and other lactose-positive coliforms, and the combination of a pH indicator such as bromocresol purple and D-trehalose for indicating the presence of Salmonella and/or Shiga toxin-producing E. coli. Other visual indicators for indicating the presence of Salmonella, E. coli, and other microorganisms are known in the art.

The microbial growth media of the invention may include one or more selective agents described below, or otherwise known in the art, for selecting for microorganisms such as Salmonella sp. and/or Shiga toxin-producing E. coli (STEC). Each selective agent may be included in an amount effective to inhibit growth of at least one non-Salmonella microorganism and/or at least one non-Shiga toxin-producing E. coli microorganism to a greater extent than Salmonella (such as Salmonella enterica) and/or Shiga toxin-producing E. coli (such as E. coli having a type selected from O157, O145, O104, O26, O111, O103, and O91). It is preferred that the one or more selective agents are present in amounts that do not substantially inhibit the growth or metabolism of Salmonella (such as Salmonella enterica) and/or Shiga toxin-producing E. coli (such as E. coli having a type selected from O157, O145, O104, O26, O111, O103, and O91).

The microbial growth media of the invention may include one or more sulfa drugs as a selective agent. The sulfa drug serves as an anti-metabolite selective agent. Sulfa drugs, also called sulfonamides or sulphonamides, are antimicrobial agents that contain the sulfonamide group. Examples of suitable sulfa drugs include aldesulfone sodium, elixir sulfanilamide, mafenide, phthalylsulfathiazole, prontosil, silver sulfadiazine, succinylsulfathiazole, sulfabenzamide, sulfacetamide, sulfacytine, sulfadiazine, sulfadicramide, sulfadimethoxine, sulfadimidine, sulfadoxine, sulfafurazole, sulfaguanidine, sulfalene, sulfamazone, sulfamerazine, sulfamethizole, sulfamethoxazole, sulfamethoxypyridazine, sulfametomidine, sulfametoxydiazine, sulfametrole, sulfamoxole, sulfanilamide, sulfaperin, sulfaphenazole, sulfapyridine, sulfaquinoxaline, sulfathiazole, sulfathiourea, sulfatolamide, and sulfisomidine, among others. Sulfathiazole is preferably included because of its toxicity to some Citrobacter spp. A preferred combination of sulfa drugs includes sulfanilamide and sulfathiazole in a ratio of about 9:1, such as in concentrations of about 0.9 g/L and 0.1 g/L, respectively. Exemplary concentration ranges of the one or more sulfa drugs include from about 0.05 g/L to about 20 g/L, such as from about 0.1 g/L to about 10 g/L or from about 0.5 g/L to about 2.0 g/L. Such concentrations refer to the total concentration of all sulfa drugs in the composition.

The microbial growth media of the invention may contain one or more surfactants as a selective agent. The surfactant may be a non-ionic surfactant, an ionic surfactant, or an amphoteric surfactant. If an ionic surfactant, the surfactant may be a cationic surfactant or an anionic surfactant. Preferred surfactants are anionic surfactants, such as aliphatic sulfates. The aliphatic sulfate may have a branched aliphatic chain or a linear aliphatic chain. Preferred aliphatic sulfates include 7-ethyl-2-methyl-4-undecanol hydrogen sulfate or sodium salt thereof (Tergitol 4; CAS No. 139-88-8) and 7-ethyl-2-methyl-4-undecyl sulfate or sodium salt thereof (NIAPROOF® 4, available under Cat. No. N1404 from Sigma-Aldrich Co., St. Louis, MO). The 7-ethyl-2-methyl-4-undecyl sulfate or a sodium salt thereof is particularly preferred. These aliphatic sulfates inhibit growth of Proteus spp. Exemplary concentration ranges of the one or more surfactants include concentrations from about 0.001 g/L to about 100 g/L, such as from about 0.01 g/L to about 10 g/L or from about 0.1 g/L to about 1 g/L.

The microbial growth media of the invention may contain one or more aminocoumarins as a selective agent. Aminocoumarins include clorobiocin, coumermycin A1, and novobiocin. Novobiocin is a preferred aminocoumarin for inclusion in the microbial growth media of the invention. Novobiocin is a Gram-positive antibacterial. Novobiocin appears to facilitate Salmonella spp. recovery in selective enrichment media, probably by inhibiting the growth of competitive microorganisms. Exemplary concentration ranges of the one or more aminocoumarins include concentrations from about 0.002 g/L to about 1 g/L, such as from about 0.004 g/L to about 0.5 g/L or from about 0.02 g/L to about 0.10 g/L.

The microbial growth media of the invention may contain cycloheximide as a selective agent. Cycloheximide is an inhibitor of protein biosynthesis in eukaryotic organisms and thereby inhibits the growth of mold and yeast. Addition of cycloheximide is useful, as some yeasts can ferment 2-deoxy-D-ribose. Exemplary concentration ranges of the cycloheximide include concentrations from about 0.001 g/L to about 1.0 g/L, such as from about 0.002 g/L to about 0.5 g/L or from about 0.01 g/L to about 0.10 g/L.

The microbial growth media of the invention may contain a supravital stain as a selective agent. As used herein, “supravital stain” refers to a stain that enters and stains living cells, such as bacteria. Such stains are toxic to certain organisms over time, some more so than others. Examples of supravital stains include gentian violet, crystal violet, brilliant green, bismark brown, safranin, methylene blue, and malachite blue, among others. Preferred supravital stains include those that are more highly toxic to non-Salmonella microorganisms and/or non-Shiga toxin-producing E. coli microorganisms than Salmonella and/or non-Shiga toxin-producing E. coli. Brilliant green is a preferred supravital stain for including the microbial growth media. Brilliant green is a trimethylaryl dye that inhibits certain non-Salmonella Gram-negative and Gram-positive bacteria. Brilliant green inhibits many commensal E. coli but surprisingly has no effect against Shiga toxin-producing E. coli at concentrations of about 1 mg/L. Brilliant green bleaches at low pH and therefore does not interfere with the pH indicator reaction. Other supravital stains, such as malachite blue, do not bleach effectively at low pH and would therefore preclude the use of a chromogenic pH indicator. Such supravital stains, however, may be used when an indication of a change in pH is not desired or needed. The supravital stain may be included in the microbial growth media at a concentration from about 0.0001 g/L to about 0.5 g/L, such as from about 0.0002 g/L to about 0.25 g/L or from about 0.001 g/L to about 0.05 g/L.

The microbial growth media of the invention may contain ascorbic acid as a selective agent. Ascorbic acid has inhibitory activity against some species of Citrobacter sp. See U.S. Pat. No. 4,279,995 to Woods et al. Ascorbic acid may be included in the microbial growth media at a concentration from about 0.05 g/L to about 20 g/L, such as from about 0.1 g/L to about 10 g/L or from about 0.5 g/L to about 2.0 g/L.

The microbial growth media of the invention may contain bromobenzoic acid as a selective agent. Bromobenzoic acid has inhibitory activity against some species of Citrobacter. See U.S. Pat. No. 4,279,995 to Woods et al. Bromobenzoic acid may be included in the microbial growth media at a concentration from about 0.001 g/L to about 1.0 g/L, such as from about 0.002 g/L to about 0.50 g/L or from about 0.01 g/L to about 0.10 g/L.

The microbial growth media of the invention may contain myricetin as a selective agent. Myricetin inhibits Enterobacter and Klebsiella spp. Brilliant green inhibits many commensal E. coli but surprisingly has no effect against Shiga toxin-producing E. coli or Salmonella at concentrations of about 1 mg/L. Myricetin may be included in the microbial growth media at a concentration from about 0.001 g/L to about 1.0 g/L, such as from about 0.002 g/L to about 0.50 g/L or from about 0.01 g/L to about 0.10 g/L. Lower concentrations may be preferred for selection of Shiga toxin-producing E. coli.

The microbial growth media of the invention may contain nitrofurantoin as a selective agent. Nitrofurantoin is 1-[[(5-Nitro-2-furanyl)methylene]amino]-2,4-imidazolidinedione and is available under Cat. No. N7878 from Sigma-Aldrich Co., St. Louis, MO. Nitrofurantoin is typically used to treat urinary tract infection, and is often used against E. coli. As shown in U.S. Pat. No. 9,518,283, nitrofurantoin is surprisingly ineffective against Shiga toxin-producing E. coli and Salmonella and can therefore be used to select for these microorganisms. The nitrofurantoin may be included in the microbial growth media at a concentration from about 0.0001 g/L to about 0.1 g/L, such as from about 0.0005 g/L to about 0.05 g/L or from about 0.001 g/L to about 0.01 g/L.

The microbial growth media of the invention may contain one or more rifamycins as a selective agent. Suitable rifamycins include rifamycins A, B, C, D, E, S, and SV as well as the rifamycin derivatives rifampicin (or rifampin), rifabutin, rifapentine, and rifalazil. Rifampicin is preferred. Exemplary concentration ranges of the rifamycin include concentrations from about 0.0001 g/L to about 0.2 g/L, such as from about 0.0002 g/L to about 0.1 g/L or from about 0.001 g/L to about 0.02 g/L.

The microbial growth media of the invention may contain one or more polyketides as a selective agent. Suitable polyketides include macrolide antibiotics such as pikromycin, erythromycin A, clarithromycin, and azithromycin; polyene antibiotics such as amphotericin; tetracycline; and doxacycline. Tetracycline and/or doxycycline are preferred. Exemplary concentration ranges of the polyketide include concentrations from about 0.0001 g/L to about 0.2 g/L, such as from 0.0002 g/L to about 0.1 g/L or from 0.001 g/L to about 0.02 g/L.

The microbial growth media of the invention may contain one or more oxazolidinones as a selective agent. Suitable oxazolidinones include linezolid (ZYVOX®, Pfizer, Inc., New York, NY), posizolid, torezolid, radezolid (RX-1741), and cycloserine. Linezolid is preferred. Exemplary concentration ranges of the oxazolidinone include concentrations from about 0.0001 g/L to about 0.2 g/L, such as from about 0.0002 g/L to about 0.1 g/L or from about 0.001 g/L to about 0.02 g/L.

Some microbial growth media of the invention comprise only one of a sulfa drug, a surfactant, an aminocoumarin, cycloheximide, a supravital stain, ascorbic acid, bromobenzoic acid, myricetin, nitrofurantoin, a rifamycin, a polyketide, or an oxazolidinone as a selective agent. Some microbial growth media of the invention comprise a sulfa drug in combination with any one, all, or subcombinations of a surfactant, an aminocoumarin, cycloheximide, a supravital stain, ascorbic acid, bromobenzoic acid, myricetin, nitrofurantoin, a rifamycin, a polyketide, and an oxazolidinone as a selective agent. Some microbial growth media of the invention comprise a surfactant in combination with any one, all, or subcombinations of a sulfa drug, an aminocoumarin, cycloheximide, a supravital stain, ascorbic acid, bromobenzoic acid, myricetin, nitrofurantoin, a rifamycin, a polyketide, and an oxazolidinone as a selective agent. Some microbial growth media of the invention comprise an aminocoumarin in combination with any one, all, or subcombinations of a sulfa drug, a surfactant, cycloheximide, a supravital stain, ascorbic acid, bromobenzoic acid, myricetin, nitrofurantoin, a rifamycin, a polyketide, and an oxazolidinone as a selective agent. Some microbial growth media of the invention comprise cycloheximide in combination with any one, all, or subcombinations of a sulfa drug, a surfactant, an aminocoumarin, a supravital stain, ascorbic acid, bromobenzoic acid, myricetin, nitrofurantoin, a rifamycin, a polyketide, and an oxazolidinone as a selective agent. Some microbial growth media of the invention comprise a supravital stain in combination with any one, all, or subcombinations of a sulfa drug, a surfactant, an aminocoumarin, cycloheximide, ascorbic acid, bromobenzoic acid, myricetin, nitrofurantoin, a rifamycin, a polyketide, and an oxazolidinone as a selective agent. Some microbial growth media of the invention comprise ascorbic acid in combination with any one, all, or subcombinations of a sulfa drug, a surfactant, an aminocoumarin, cycloheximide, a supravital stain, bromobenzoic acid, myricetin, nitrofurantoin, a rifamycin, a polyketide, and an oxazolidinone as a selective agent. Some microbial growth media of the invention comprise bromobenzoic acid in combination with any one, all, or subcombinations of a sulfa drug, a surfactant, an aminocoumarin, cycloheximide, a supravital stain, ascorbic acid, myricetin, nitrofurantoin, a rifamycin, a polyketide, and an oxazolidinone as a selective agent. Some microbial growth media of the invention comprise myricetin in combination with any one, all, or subcombinations of a sulfa drug, a surfactant, an aminocoumarin, cycloheximide, a supravital stain, ascorbic acid, bromobenzoic acid, nitrofurantoin, a rifamycin, a polyketide, and an oxazolidinone as a selective agent. Some microbial growth media of the invention comprise nitrofurantoin in combination with any one, all, or subcombinations of a sulfa drug, a surfactant, an aminocoumarin, cycloheximide, a supravital stain, ascorbic acid, bromobenzoic acid, myricetin, a rifamycin, a polyketide, and an oxazolidinone as a selective agent. Some microbial growth media of the invention comprise a rifamycin in combination with any one, all, or subcombinations of a sulfa drug, a surfactant, an aminocoumarin, cycloheximide, a supravital stain, ascorbic acid, bromobenzoic acid, myricetin, nitrofurantoin, a polyketide, and an oxazolidinone as a selective agent. Some microbial growth media of the invention comprise a polyketide in combination with any one, all, or subcombinations of a sulfa drug, a surfactant, an aminocoumarin, cycloheximide, a supravital stain, ascorbic acid, bromobenzoic acid, myricetin, nitrofurantoin, a rifamycin, and an oxazolidinone as a selective agent. Some microbial growth media of the invention comprise an oxazolidinone in combination with any one, all, or subcombinations of a sulfa drug, a surfactant, an aminocoumarin, cycloheximide, a supravital stain, ascorbic acid, bromobenzoic acid, myricetin, nitrofurantoin, a rifamycin, and a polyketide as a selective agent.

The microbial growth media of the invention may also contain one or more efflux pump inhibitors (EPIs). As used herein, “efflux pump inhibitor” refers to any agent capable of inhibiting a bacterial efflux pump. The EPI preferably increases the toxicity of selective agents in non-Salmonella and non-Shiga toxin-producing E. coli microorganisms.

Phylogenetically, bacterial antibiotic efflux pumps belong to five superfamilies (see reviews (Li XZ, Nikaido H. Efflux-mediated drug resistance in bacteria. Drugs 2004; 64:159-204) (Paulsen I T. Multidrug efflux pumps and resistance: regulation and evolution. Curr Opin Microbiol 2003; 6:446-51) (Saier M H Jr. Tracing pathways of transport protein evolution. Mol Microbiol 2003; 48:1145-56)), namely: (i) ABC (ATP-binding cassette), which are primary active transporters energized by ATP hydrolysis; (ii) SMR [small multidrug resistance subfamily of the DMT (drug/metabolite transporters) superfamily]; (iii) MATE [multi-antimicrobial extrusion subfamily of the MOP (multidrug/oligosaccharidyl-lipid/polysaccharide flippases) superfamily]; (iv) MFS (major facilitator superfamily); and (v) RND (resistance/nodulation/division superfamily), which are all secondary active transporters driven by ion gradients. The MFS and RND pumps are the most abundant. The MFS pumps are found in both Gram-positive and Gram-negative bacteria, and are characterized by a relative narrow spectrum, recognizing usually one or sometimes a few antibiotic classes; the RND pumps are found exclusively in Gram-negative bacteria and display an extremely wide spectrum of substrates (poly-selectivity), including not only several classes of antibiotics, but also antiseptic compounds, dyes, or detergents. See: (1) Levy S B. Active efflux, a common mechanism for biocide and antibiotic resistance. J Appl Microbiol 2002; 92 Suppl:65-71; (2) Li XZ, Nikaido H. Efflux-mediated drug resistance in bacteria. Drugs 2004; 64:159-204; (3) Lomovskaya O, Totrov M. Vacuuming the periplasm. J. Bacteriol 2005; 187:1879-83; (4) Poole K. Efflux-mediated antimicrobial resistance. J Antimicrob Chemother 2005; 56:20-51; (5) Van Bambeke F, Glupczynski Y, Plesiat P, et al. Antibiotic efflux pumps in prokaryotic cells: occurrence, impact on resistance and strategies for the future of antimicrobial therapy. J Antimicrob Chemother 2003; 51:1055-65; (6) Koronakis V. TolC—the bacterial exit duct for proteins and drugs. FEBS Lett 2003; 555:66-71; and (7) Piddock U. Clinically relevant chromosomally encoded multidrug resistance efflux pumps in bacteria. Clin Microbiol Rev 2006; 19:382-402.

Suitable EPIs that may be included in the microbial growth media of the invention include phenothiazines (see Molnar J, Hever A, Fakla I, et al. Inhibition of the transport function of membrane proteins by some substituted phenothiazines in E. coli and multidrug resistant tumor cells. Anticancer Res 1997; 17:481-6), phenylpiperidines (see Kaatz G W, Moudgal V V, Seo S M, et al. Phenylpiperidine selective serotonin reuptake inhibitors interfere with multidrug efflux pump activity in Staphylococcus aureus. Int J Antimicrob Agents 2003; 22:254-61), tetracycline analogs (Nelson M L, Park B H, Andrews J S, et al. Inhibition of the tetracycline efflux antiport protein by 13-thio-substituted 5-hydroxy-6-deoxytetracyclines. J Med Chem 1993; 36:370-7; and Nelson M L, Park B H, Levy S B. Molecular requirements for the inhibition of the tetracycline antiport protein and the effect of potent inhibitors on the growth of tetracycline-resistant bacteria. J Med Chem 1994; 37:1355-61.), aminoglycoside analogs (Frangoise Van Bambeke, Jean-Marie Pages and Ving J. Lee. Inhibitors of Bacterial Efflux Pumps as Adjuvants in Antibiotic Treatments and Diagnostic Tools for Detection of Resistance by Efflux. Recent Patents on Anti-Infective Drug Discovery, 2006, 1, 157-175), fluoroquinolone analogs (Frangoise Van Bambeke, Jean-Marie Pages and Ving J. Lee. Inhibitors of Bacterial Efflux Pumps as Adjuvants in Antibiotic Treatments and Diagnostic Tools for Detection of Resistance by Efflux. Recent Patents on Anti-Infective Drug Discovery, 2006, 1, 157-175), quinoline derivatives (Mahamoud A, Chevalier J, Davin-Regli A, et al. Quinolone derivatives as promising inhibitors of antibiotic efflux pump in multidrug resistant Enterobacter aerogenes. Curr Drug Targets 2006; 7:843-7), peptidomimetics (Lomovskaya O, Bostian K A. Practical applications and feasibility of efflux pump inhibitors in the clinic—a vision for applied use. Biochem Pharmacol 2006; 71:910-18), pyridopyrimidines (Nakayama K, Ishida Y, Ohtsuka M, et al. MexAB-OprM-specific efflux pump inhibitors in Pseudomonas aeruginosa. Part 1: discovery and early strategies for lead optimization. Bioorg Med Chem Lett 2003; 13:4201-4; Nakayama K, Ishida Y, Ohtsuka M, et al. MexAB-OprM specific efflux pump inhibitors in Pseudomonas aeruginosa. Part 2: achieving activity in vivo through the use of alternative scaffolds. Bioorg Med Chem Lett 2003; 13:4205-8; Nakayama K, Kawato H, Watanabe J, et al. MexAB-OprM specific efflux pump inhibitors in Pseudomonas aeruginosa. Part 3: Optimization of potency in the pyridopyrimidine series through the application of a pharmacophore model. Bioorg Med Chem Lett 2004; 14:475-9; Nakayama K, Kuru N, Ohtsuka M, et al. MexAB-OprM specific efflux pump inhibitors in Pseudomonas aeruginosa. Part 4: Addressing the problem of poor stability due to photoisomerization of an acrylic acid moiety. Bioorg Med Chem Lett 2004; 14:2493-7; and Yoshida K, Nakayama K, Kuru N, et al. MexAB-OprM specific efflux pump inhibitors in Pseudomonas aeruginosa. Part 5: Carbon-substituted analogues at the C-2 position. Bioorg Med Chem 2006; 14:1993-2004), arylpiperidines (Thorarensen A, Presley-Bodnar A L, Marotti K R, et al. 3-Arylpiperidines as potentiators of existing antibacterial agents. Bioorg Med Chem Lett 2001; 11:1903-6), and arylpiperazines (Bohnert J A, Kern W V. Selected arylpiperazines are capable of reversing multidrug resistance in Escherichia coli overexpressing RND efflux pumps. Antimicrob Agents Chemother 2005; 49:849-52; Schumacher A, Steinke P, Bohnert J A, et al. Effect of 1-(1-naphthylmethyl)-piperazine, a novel putative efflux pump inhibitor, on antimicrobial drug susceptibility in clinical isolates of Enterobacteriaceae other than Escherichia coli. J Antimicrob Chemother 2006; 57:344-8; Kern W V, Steinke P, Schumacher A, et al. Effect of 1-(1-naphthylmethyl)-piperazine, a novel putative efflux pump inhibitor, on antimicrobial drug susceptibility in clinical isolates of Escherichia coli. J Antimicrob Chemother 2006; 57:339-43; Pannek S, Higgins P G, Steinke P, et al. Multidrug efflux inhibition in Acinetobacter baumannii: comparison between 1-(1-naphthylmethyl)-piperazine and phenyl-arginine-beta-naphthylamide. J Antimicrob Chemother 2006; 57:970-4.). See also Mahamoud, A. et al. Antibiotic efflux pumps in Gram-negative bacteria: the inhibitor response strategy. J. Antimicrob. Chemoth. 59(6):1223-1229. 2007.

Suitable phenothiazines include promethazine and 3,7,8,-trihydroxy-, 7,8-dihydroxy, 7,8-diacetoxy-, 7,8dimetoxy-, 7-semicarbazone-, and 5-oxo-chlorpromazine derivatives, among others. Suitable phenylpiperidines include the paroxetine isomer NNC 20-7052, among others. Suitable tetracycline analogs include 13-(alkylthio) and 13-(arylthio) derivatives of 5-hydroxy-6-deoxytetracycline, among others. Suitable aminoglycoside analogs include the aminoglycosides described in U.S. Pat. No. 7,829,543, among others. Suitable fluoroquinolone analogs include those described in WO0209758A2 and WO0209758A3, among others. Suitable quinoline derivatives include alkylamino-, alkyl-alkoxy-, thioalkoxy-, and halo-quinoline derivatives (e.g., chloroquinoline derivatives) and those having piperidinoethyl chains, among others. A preferred quinoline derivative is 4-chloroquinoline. Suitable peptidomimetics include MC-207 110 or phenylalanine arginyl 0-naphthylamide (PAON), and derivatives thereof, among others. Suitable arylpiperidines include 3-arylpiperidine derivatives, among others. Suitable arylpiperazines include arylpiperazines, including 1-(1-naphthylmethyl)piperazine and others.

The EPIs are preferably selected from the group consisting of arylpiperazines, such as 1-(1-naphthylmethyl)piperazine (NMP; CAS No. 40675-81-8; available as Cat. No. 651699, Sigma-Aldrich Co., St. Louis, MO), and quinoline derivatives, such as 4-chloroquinoline (4-CQ; CAS No. 611-35-8; available as Cat. No. C70509, Sigma-Aldrich Co., St. Louis, MO). The NMP and 4-CQ may be included individually or together.

Combinations of an EPI with polyketide, rifamycin, or oxazolidinone antibiotics can increase the activity of these antibiotics against Enterobacteriacae and other microorganisms without substantially affecting growth and fermentation of Salmonella species and/or Shiga toxin-producing E. coli strains. Accordingly, the one or more selective agents and the efflux pump inhibitor are present in the microbial growth media of the invention in amounts effective to inhibit growth of at least one non-Salmonella species and/or at least one non-Shiga toxin-producing E. coli strain to a greater extent than Salmonella species (such as Salmonella enterica) and/or Shiga toxin-producing E. coli strains. It is preferred that the combination of the one or more selective agents and the efflux pump inhibitor are present in an amount that does not substantially affect the growth or metabolism of Salmonella species (such as Salmonella enterica) and/or Shiga toxin-producing E. coli strains.

The microbial growth media described herein can be provided in a hydrated form, such as in the form of a liquid or gel-like (e.g., agar) medium, or in a dried form. If in a dried form, the components are preferably present in a proportion such that addition of water or other solvents provides each of the components within the concentration ranges described above. In addition, the microbial growth media, whether in died or hydrated form, may be provided in separate combinations, e.g., basal media and one or more supplements.

Concentrations other than those explicitly described herein, such as above and below the stated ranges, are included in the invention.

Methods of the invention include methods of growing a microorganism. The methods comprise culturing a sample suspected of containing the microorganism in any growth medium of the invention. The microorganism is preferably a bacterium, such as a Gram-negative bacterium, such as E. coli or Salmonella. The E. coli is preferably STEC.

The methods of the invention preferably result in enhanced microbial growth stimulating activity compared to identical methods with media lacking the microbial growth-stimulating protein but otherwise identical to the microbial growth media. The enhanced microbial growth stimulating activity is preferably for a bacterium, such as a Gram-negative bacterium, such as E. coli or Salmonella. The E. coli is preferably STEC.

The microbial growth media of the invention can be used to select for Salmonella and/or Shiga toxin-producing E. coli from other microorganisms, the latter including Citrobacter spp. (e.g., Citrobacter freundii, Citrobacter koseri, etc.), non-Shiga toxin-producing E. coli, and/or commensal E. coli generally. The microbial growth media of the invention can be used for detecting Salmonella and/or Shiga toxin-producing E. coli. Specifically, the microbial growth media of the invention can be used for cultivating or selecting for Salmonella (including Salmonella enterica); cultivating or selecting for Shiga toxin-producing E. coli (including E. coli having a type selected from O157, O145, O104, O26, O111, O103, and O91); or co-cultivating or co-selecting for Salmonella (including Salmonella enterica) and Shiga toxin-producing E. coli (including E. coli having a type selected from O157, O145, O104, O26, O111, O103, and O91).

Some methods of the invention comprise culturing a sample suspected of containing the microorganism in a microbial growth medium comprising a growth-stimulating amount of phosphoglucomutase.

In some versions, the growth medium comprises a combination of a base medium and a composition comprising the phosphoglucomutase. The composition can be any microbial growth-stimulating protein composition described herein. The base medium can comprise any one or more medium components described herein, in any combination.

In some versions, the composition is devoid of lipid or contains lipid in an amount less than 20% w/w, less than 15% w/w, less than 10% w/w, less than 5% w/w, less than 1% w/w, less than 0.75% w/w, less than 0.5% w/w, less than 0.25% w/w, less than 0.1% w/w, less than 0.075% w/w, less than 0.05% w/w, less than 0.025% w/w, less than 0.01% w/w, less than 0.0075% w/w, less than 0.005% w/w, less than 0.0025% w/w, less than 0.001% w/w, less than 0.00075% w/w, less than 0.0005% w/w, less than 0.00025% w/w, or less than 0.0001% w/w. “Lipid” in this context refers to total lipid. Methods for measuring total lipid concentration in a sample are well known in the art. See, e.g., Shahidi, Fereidoon. (2001) Extraction and Measurement of Total Lipids. Current Protocols in Food Analytical Chemistry, Volume 7, Issue 1, D1.1.1-D1.1.11, John Wiley & Sons. https://doi.org/10.1002/0471142913.fad0101s07.

In some versions, the method comprises combining the base medium and the composition. In some versions, the composition is in the form of a liquid prior to combining with the base medium. In some versions, the composition is in the form of a solid prior to combining with the base medium. In some versions, the composition is in the form of a powder prior to combining with the base medium.

In some versions, the growth medium is devoid of lipid or contains lipid in an amount less than 20% w/w, less than 15% w/w, less than 10% w/w, less than 5% w/w, less than 1% w/w, less than 0.75% w/w, less than 0.5% w/w, less than 0.25% w/w, less than 0.1% w/w, less than 0.075% w/w, less than 0.05% w/w, less than 0.025% w/w, less than 0.01% w/w, less than 0.0075% w/w, less than 0.005% w/w, less than 0.0025% w/w, less than 0.001% w/w, less than 0.00075% w/w, less than 0.0005% w/w, less than 0.00025% w/w, or less than 0.0001% w/w. “Lipid” in this context refers to total lipid. Methods for measuring total lipid concentration in a sample are well known in the art. See, e.g., Shahidi, Fereidoon. (2001) Extraction and Measurement of Total Lipids. Current Protocols in Food Analytical Chemistry, Volume 7, Issue 1, D1.1.1-D1.1.11, John Wiley & Sons. https://doi.org/10.1002/0471142913.fad0101s07.

In some versions, the phosphoglucomutase comprises a phosphoglucomutase other than bovine phosphoglucomutase. In some versions, the phosphoglucomutase comprises yeast phosphoglucomutase.

Some methods of the invention further comprise detecting presence of the microorganism. Methods for detecting various Salmonella species and E. coli such as STEC are provided in U.S. Pat. Nos. 9,518,283 and 9,029,118.

Kits for use of the microbial growth media of the invention may include any combination of components described herein.

In some versions, the microbial growth-stimulating protein compositions of the invention are prepared using a step of incubating an extraction liquid comprising water and a surfactant with an animal meat for a time sufficient to extract microbial growth-stimulating protein from the animal meat into the extraction liquid to thereby generate a conditioned composition.

In some versions of the invention, the extraction liquid can comprise the water in amount of at least 30% w/w, such as at least about 35% w/w, at least about 40% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w.

Surfactants are amphiphilic compounds that comprise a hydrophilic head and a hydrophobic tail. The hydrophilic head may comprise a polar, nonionic head group or an ionic head group. The ionic head group may be an anionic head group, a cationic head group, or a zwitterionic (amphoteric) head group.

Nonionic surfactants are surfactants that have non-ionic head groups. The nonionic head groups may include hydroxyl groups or other polar groups. Examples of nonionic surfactants include long chain alcohols, such as cetyl alcohol, stearyl alcohol, cetostearyl alcohol (consisting predominantly of cetyl and stearyl alcohols), and oleyl alcohol; polyoxyethylene glycol alkyl ethers (Brij), such as those having the formula CH₃—(CH₂)₁₀₋₁₆—(O—C₂H₄)₁₋₂₅—OH, including octaethylene glycol monododecyl ether and pentaethylene glycol monododecyl ether, among others; polyoxypropylene glycol alkyl ethers, such as those having the formula CH₃—(CH₂)₁₀₋₁₆—(O—C₃H₆)₁₋₂₅—O; glucoside alkyl ethers, such as those having the formula CH₃—(CH₂)₁₀₋₁₆—(O-Glucoside)₁₋₃-OH, including decyl glucoside, lauryl glucoside, and octyl glucoside, among others; polyoxyethylene glycol octylphenol ethers, such as those having the formula C₈H₁₇—(C₆H₄)—(O—C₂H₄)₁₋₂₅—OH, including Triton X-100, among others; polyoxyethylene glycol alkylphenol ethers, such as those having the formula C₉H₁₉—(C₆H₄)—(O—C₂H₄)₁₋₂₅—OH, including nonoxynol-9, among others; glycerol alkyl esters, such as glyceryl laurate, among others; polyoxyethylene glycol sorbitan alkyl esters, such as polysorbate, among others; sorbitan alkyl esters, such as Spans, among others; cocamide MEA; cocamide DEA; codecyldimethylamine oxide; block copolymers of polyethylene glycol and polypropylene glycol, such as poloxamers, among others; and polyethoxylated tallow amine (POEA).

Anionic surfactants are surfactants that have anionic head groups. The anionic head groups may include sulfate, sulfonate, phosphate, and/or carboxylate groups, among others. Examples of anionic surfactants include alkyl sulfates, such as ammonium lauryl sulfate, sodium lauryl sulfate (SDS, sodium dodecyl sulfate), alkyl-ether sulfates such as sodium laureth sulfate, and sodium myreth sulfate, among others. Examples of anionic surfactants also include sulfonates, such as sodium dodecyl sulfonate, dioctyl sodium sulfosuccinate, perfluorooctanesulfonate (PFOS), perfluorobutanesulfonate, and linear alkylbenzene sulfonates (LABs), among others. Carboxylates are preferred surfactants. Carboxylates comprise alkyl carboxylates, such as fatty acids and salts thereof. Examples of carboxylates include sodium stearate, sodium lauroyl sarcosinate, and carboxylate-based fluorosurfactants, such as perfluorononanoate, and perfluorooctanoate (PFOA or PFO). Preferred anionic surfactants include cocoyl isethionate, sodium dodecylbenzinesulfonate, and sodium isethionate.

Cationic surfactants are surfactants that have cationic head groups. The cationic head groups may include pH-dependent primary, secondary, or tertiary amines and permanently charged quaternary ammonium cations, among others. Primary amines become positively charged at pH<10, secondary amines become positively charged at pH<4. An example of a pH-dependent amine is octenidine dihydrochloride. Permanently charged quaternary ammonium cations include alkyltrimethylammonium salts, such as cetyl trimethylammonium bromide (CTAB, hexadecyl trimethyl ammonium bromide), cetyl trimethylammonium chloride (CTAC), cetylpyridinium chloride (CPC), benzalkonium chloride (BAC), benzethonium chloride (BZT), 5-Bromo-5-nitro-1,3-dioxane, dimethyldioctadecylammonium chloride, cetrimonium bromide, and dioctadecyldimethylammonium bromide (DODAB), among others.

Zwitterionic (amphoteric) surfactants are surfactants that have zwitterionic head groups. Zwitterionic head groups include both cationic and anionic centers. The cationic center may be based on primary, secondary, or tertiary amines, quaternary ammonium cations, or others. The anionic part may include sulfonates, as in CHAPS (3-[(3-Cholamidopropyl)dimethylammonio]-1-propanesulfonate), or sultaines, as in cocamidopropyl hydroxysultaine. Other examples of zwitterionic head groups include betaines, such as cocamidopropyl betaine, and choline-phosphates, such as those occurring in lecithin, among others.

For ionic head groups, the counter-ion can be monoatomic/inorganic or polyatomic/organic. Monoatomic/inorganic cationic counter-ions include metals, such as the alkali metals, alkaline earth metals, and transition metals. Monoatomic/inorganic anionic counter-ions include the halides, such as chloride (Cl—), bromide (Br—), and iodide (I—). Polyatomic/organic cationic counter-ions include ammonium, pyridinium, and triethanolamine (TEA), among others. Polyatomic/organic anionic counter-ions include tosyls, trifluoromethanesulfonates, and methylsulfate, among others.

The hydrophobic tail of the surfactant may include a linear, branched, or aromatic hydrocarbon chain. The hydrocarbon chain may have any number of carbon atoms suitable to render it hydrophobic. The carbon atoms may be saturated, unsaturated, straight-chained, branched, or cyclic. The hydrocarbon chain may be substituted with one or more heteroatoms.

Preferred surfactants for use in the extraction liquid are anionic surfactants, such as aliphatic sulfates. The aliphatic sulfate may have a branched aliphatic chain or a linear aliphatic chain. Preferred aliphatic sulfates include 7-ethyl-2-methyl-4-undecanol hydrogen sulfate or sodium salt thereof (Tergitol 4; CAS No. 139-88-8) and 7-ethyl-2-methyl-4-undecyl sulfate or sodium salt thereof (NIAPROOF® 4, available under Cat. No. N1404 from Sigma-Aldrich Co., St. Louis, MO). The 7-ethyl-2-methyl-4-undecyl sulfate or a sodium salt thereof is particularly preferred.

Exemplary amounts of the surfactant included in the extraction liquid include from about 0.001 g/L to about 100 g/L, such as from about 0.01 g/L to about 10 g/L or from about 0.1 g/L to about 1 g/L.

The extraction liquid can further include a divalent cation. Exemplary divalent cations include Mg²⁺ and Ca²⁺. The divalent cation may be added to or included in the form of a salt, such as an inorganic salt. Exemplary inorganic salts are described below with reference to the microbial growth media of the invention. Exemplary amounts of the inorganic salt included in the extraction liquid are from about 0.01 g/L to about 50 g/L, such as from about 0.1 g/L to about 40 g/L, from about 0.5 g/L to about 35 g/L, or from about 1 g/L to about 15 g/L.

The extraction liquid in some versions is devoid of an amount of a carbon and nitrogen source sufficient to support microbial growth. Carbon and nitrogen sources are described elsewhere herein. Exemplary microorganisms that can be tested for microbial growth in this regard can an E. coli such as a STEC or a Salmonella species. Exemplary methods for testing for growth of a microbe such as E. coli or Salmonella are provided in the following examples.

The Extraction liquid in some versions comprises no more than: 0.5 g/L sulfanilamide; 0.05 g/L sulfathiazole; 0.01 g/L novobiocin; 0.025 g/L cycloheximide; 0.5 g/L ascorbic acid; 0.0025 g/L myricetin; 2.5 g/L 2-deoxy-D-ribose; 0.0024 g/L doxycycline; 0.02 g/L naphthylmethyl piperazine; 10 g/L peptone; or any combination of any of the foregoing. “Comprises no more than” in this context refers to comprising a given component in an amount less than the aforementioned amount or being completely devoid of the given component.

The animal meat can comprise meat from any animal. Exemplary animals include aquiline, asinine, bovine, cancrine, canine, cervine, corvine, equine, elapine, elaphine, feline, hircine, leonine, leporine, lupine, murine, pavonine, piscine, porcine, rusine, serpentine, ursine, volucrine, and vulpine animals. “Meat” refers to any muscle and offal. Exemplary types of muscle include skeletal muscle, cardiac muscle, and smooth muscle. “Offal” refers to non-muscle tissues or organs of the animal body. Exemplary types of offal include liver, tongue, intestines, stomach, rumen, tripe (i.e., from the reticulum or rumen), glands (e.g., pancreas, kidney, and thymus), heart, lung, and brain. The meat can be in any of a number of forms. The meat can be raw or cooked. The meat can be processed or unprocessed. Exemplary forms of processed meat include ground meat, sliced meat, and minced meat.

The extraction liquid is incubated with the animal meat for a time sufficient to extract microbial growth-stimulating protein from the animal meat into the extraction liquid. “Microbial growth-stimulating protein” refers to protein that confers an increased rate of growth of a microbe as indicated by an increased slope in a growth curve. The microbe can be an E. coli such as a STEC or a Salmonella species. Exemplary methods for testing for a growth curve of a microbe such as E. coli or Salmonella are provided in the following examples.

The extraction of microbial growth-stimulating protein from the animal meat into the extraction liquid results in a conditioned composition. The conditioned composition may comprise, along with the microbial growth-stimulating protein and the conditioned composition, other components extracted from the animal meat. Such components may comprise non-microbial growth-stimulating protein, nucleic acids, carbohydrates, sugars, vitamins, minerals, or other inorganic or organic biomolecules.

Once the conditioned liquid is generated, the microbial growth-stimulating protein can be separated from at least a portion of at least one other component of the conditioned composition. Such separation at least partially purifies or concentrates the microbial growth-stimulating protein. The at least one other component of the conditioned composition can comprise any component of the conditioned composition other than the microbial growth-stimulating protein. At the time of separation, the component can be included in the conditioned composition itself or a downstream, processed composition generated from adding or removing other components to or from the original conditioned composition. Exemplary components include the water of the extraction liquid, the divalent cation (or salt) of the extraction liquid, any other component of the extraction liquid, and any component extracted from the animal meat other than the microbial growth-stimulating protein. Exemplary separation methods for separating the microbial growth-stimulating protein from at least a portion of at least one other component of the conditioned composition include, filtration, precipitation, chromatography, centrifugation, chelation, extraction, flotation, electrophoresis, and adsorption, among others. The separation of the component can be complete or partial.

In some versions, the separating comprises precipitating the microbial growth-stimulating protein. The microbial growth-stimulating protein can be precipitated from the conditioned composition or any downstream processed composition thereof. An exemplary precipitation method includes protein precipitation. “Protein precipitation” refers to any method that precipitates protein from a solution. Exemplary methods include salting out and salting in, isoelectric precipitation, precipitation with miscible solvents (e.g. ethanol or methanol), flocculation by polyelectrolytes (e.g., alginate, carboxymethycellulose, polyacrylic acid, tannic acid, and polyphosphates), and precipitating with polyvalent metallic ions (e.g., Ca²⁺, Mg²⁺, Mn²⁺ or Fe²⁺). An exemplary method of salting out is ammonium sulfate precipitation. Separation of the precipitate from the precipitated liquid, can occur through filtration, centrifugation followed by aspiration and/or decanting, or other methods known in the art. In some versions, the precipitate is desalted to generate a desalted precipitate. The precipitate can be desalted through filtration with a filter having a molecular weight cutoff of 50 kDa or less, as described above.

In some versions, the microbial growth-stimulating protein is precipitated with an amount of ammonium sulfate from about 1% ammonium sulfate saturation to about 95% ammonium sulfate saturation, such as from about 1%, about 5%, about 10%, about 15%, about 20%, or about 30% to about 50%, about 55%, about 60%, about 65%, about 70%, about 75%, about 80%, about 90%, or about 95% ammonium sulfate saturation.

In some versions, the separating comprises precipitating a component of the conditioned composition from the microbial growth-stimulating protein. In some versions, this can occur through precipitating the component with an amount of ammonium sulfate from about 1% ammonium sulfate saturation to about 30% ammonium sulfate saturation, such as from about 1%, about 5%, about 10%, or about 15% to about 10%, about 15%, about 20%, or about 30% ammonium sulfate saturation. Precipitating a given component from the microbial growth-stimulating protein can occur in combination with precipitating the microbial growth-stimulating protein from another given component. These two processes of separation can occur in either order. Examples of performing these processes in combination are provided with the ammonium sulfate cuts described elsewhere herein.

In some versions, the separating comprises filtering the microbial growth-stimulating protein from the other component of the conditioned composition. This can be performed, for example, with a filter having a molecular weight cutoff from about 1 kDa to about 60 kDa, such as from about 1 kDa, about 5 kDa, 10 kDa, 15 kDa, 20 kDa, 25 kDa, 30 kDa, 35 kDa, 40 kDa, 45 kDa, 50 kDa, or 55 kDa to about 10 kDa, 15 kDa, 20 kDa, 25 kDa, 30 kDa, 35 kDa, 40 kDa, 45 kDa, 50 kDa, 55 kDa, or 60 kDa. The microbial growth-stimulating protein in such a case is comprised in the retentate and is separated from components comprised in the permeate (filtrate).

In some versions the separating comprises filtering the other component of the conditioned composition from the microbial growth-stimulating protein. This can be performed, for example, with a filter having a molecular weight cutoff from about 100,000 kDa to about 300,000 kDa, such as a molecular weight cutoff from about 100,000 kDa, about 125,000 kDa, about 150,000 kDa, about 175,000 kDa, about 200,000 kDa, about 225,000 kDa, about 250,000 kDa, or about 275,000 kDa to about 125,000 kDa, about 150,000 kDa, about 175,000 kDa, about 200,000 kDa, about 225,000 kDa, about 250,000 kDa, about 275,000 kDa, or about 300,000 kDa. The microbial growth-stimulating protein in such a case is comprised in the permeate (filtrate) and is separated from the components comprised in the retentate.

In some versions, the filtration is facilitated by pressure, gravity, and/or an electric field. In some versions, the filtration is facilitated by diffusion. The terms “filtrate” and “permeate” are used herein interchangeably. In some versions, the retentate is in the form of a solid (e.g. in vacuum filtration). In some versions, the retentate is in the form of a liquid solution or suspension (e.g., in dialysis).

Before use, the microbial growth-stimulating protein is preferably sterilized. The sterilizing can be performed using a number of methods. Exemplary methods include filter sterilization (e.g., with a pore diameter of 0.03 μm to about 10 μm, such as about 0.2 μm), radiation sterilization (e.g., using ultraviolet light, X-rays, gamma rays), and chemical sterilization (e.g., ethylene oxide and carbon-dioxide gas).

Microbial growth-stimulating protein compositions prepared with precipitation can include high-molecular weight aggregates that can foul filter-sterilization membranes. Thus, it is preferred to remove these aggregates, e.g., with a filter having a molecular weight cutoff from about 100,000 kDa to about 300,000 kDa as described above, prior to filter sterilization.

The microbial growth-stimulating protein compositions of the invention can be provided in any of a number of forms. Exemplary forms include liquid, solid, and semi-solid forms. Non-limiting examples of liquid forms include the conditioned composition of the invention and any liquid retentates, resolubilized precipitates, or liquid media containing the microbial growth-stimulating protein. Non-limiting examples of solid forms include any dried, lyophilized, vacuum-filtered, gravity-filtered, or precipitated forms of the microbial growth-stimulating protein. The solid forms can include, for example, powders, crystals, or a combination thereof. Non-limiting examples of semi-solid forms include microbial growth plates, such as agar plates, containing the microbial growth-stimulating protein.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure from 1 to 10 should be construed as supporting a range from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1. Identification of Phosphoglucomutase from Ground Beef Extract as an Enteropathogen Growth Stimulating Factor

### Background

Food borne illness linked to Shiga toxin-producing Escherichia coli (STEC) and Salmonella enterica is on-going problem in the United States. United States Department of Agriculture (USDA) Food Safety and Inspection Service (FSIS) recalls involving these pathogens were reported in 2019 and 2020 in various food stuffs [1, 2]. The US Food and Drug Administration (FDA) initiated recalls during the same period involved cantaloupe [3], cinnamon apple chips [4], peaches [5] and flour [6]. The frequency and breadth of food stuffs contaminated with STEC and Salmonella demonstrate that there are challenges in the available testing methodologies to properly assess the food safety systems producing these food products. The challenges in food testing methodologies may permit contaminated food to enter the national food supply.

E. coli is usually a harmless bacterium living in the gastrointestinal tract of humans and other mammals. There are different serotypes of E. coli that have acquired bacterial Shiga toxin genes, originally arising in Shigella. The most prominent STEC associated with severe human disease in the US is E. coli O157:H7. This serotype is associated with cattle, its natural reservoir and can contaminate beef products during harvest and processing. Six other serogroups of STEC (O26, O45, O103, O111, O121 and O145) are responsible for about three-quarters of non-O157 STEC illness in the US [30]. These multiple serotypes and serogroups of pathogenic E. coli can be indistinguishable from the harmless E. coli of the gastrointestinal tract and thus pose a challenge for detection and isolation. Likewise, the CDC has identified Salmonella serotypes Enteritids, Newport, Typhimurium, and Javiana as the most common serotypes causing reportable Salmonellosis [31]. Although these are the most frequently reported Salmonella serotypes overall, their attribution to various food stuffs varies [29], with serotypes Typhimurium and Newport most commonly attributed to beef, while Enteritidis is attributed to chicken and eggs, and less common serotypes like Heidelberg attributed to turkey products. Even uncommon Salmonella serotypes such as Tennessee have been observed to emerge as significant outbreak strains, as occurred in 2006 associated with peanut butter [31].

The technical challenges of distinguishing these gram-negative pathogens from harmless gastrointestinal coliforms has been rendered substantially less difficult with the advent of PCR screening methods that target specific portions of the E. coli O157:H7 genome or common virulence factors such as the Shiga toxin gene (stx) present in STEC [7]. Similar molecular methods targeting virulence markers such as the invasion gene (invA) of Salmonella allow detection of most Salmonella serotypes [8]. Regardless of the detection method used, food samples contaminated by STEC and Salmonella must be enriched in a broth medium that increases the concentration of the pathogen to a detectable level, which is approximately 4 to 5 log₁₀ CFU/mL for most methods. Reaching this effective level can be challenging due to the out growth of other naturally present contaminating flora [32]. Numerous methods are used to enhance target pathogen growth such as incubation at restrictive temperatures (e.g., 42° C.) or inclusion of various antimicrobial compounds [9, 10, 17].

In a previous publication we detailed the development of a highly selective enrichment medium for detection and isolation of STEC and Salmonella from ground beef [9]. The media was shown to substantially reduce the complexity of the methods described in the USDA FSIS Microbiological Laboratory Guidebook (MLG) [10]. This was achieved by utilization of selective antimicrobials and inclusion of an efflux pump inhibitor that reduced the growth of background microbiological flora in the food matrix. While the medium was selective, during further validation experiments it was observed to be only effective in beef products rather than all food matrices tested. It was hypothesized that a component inherent in meat was enhancing the growth of STEC and Salmonella. In this example we describe the studies that isolated and characterized the molecular nature of the growth stimulating factor present in ground beef.

### Materials and Methods

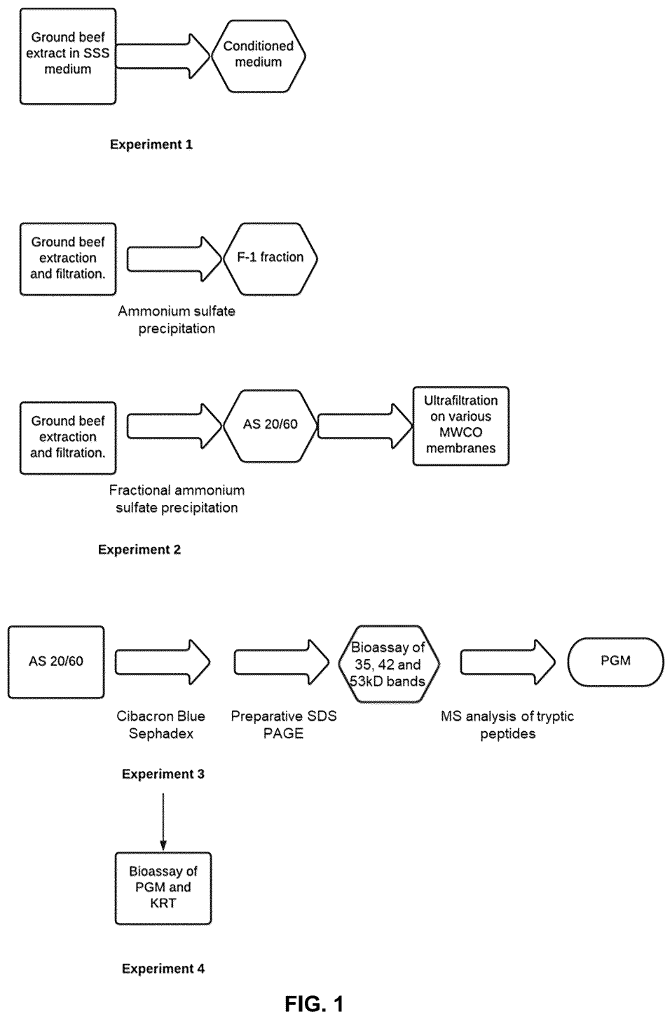

Approach: The testing of our hypothesis was addressed through the following four experiments: Experiment 1: Media conditioned with ground beef extract was compared to media containing traditional powdered beef extract supplement as control to verify STEC and Salmonella growth stimulation was directly related to the presence of fresh ground beef. Experiment 2: Ammonium sulfate precipitation and fractionation, and molecular weight fractionation of ground beef extracts were performed to determine if a specific fraction contained the growth stimulating activity. Experiment 3: Identification of compound(s) in the active ammonium sulphate fraction was performed by affinity chromatography and preparative SDS-polyacrylamide gel electrophoresis followed by mass spectral analysis of suspect tryptic peptides. Experiment 4: Commercial sourced biomolecules were obtained and tested for growth stimulating activity to confirm the identity of the active compound(s) (FIG. 1).

Media and media ingredients. Tryptic Soy Broth (TSB), MI (MUG: methylumbelliferyl-beta-D-galactopyranoside; IBDG: Inoxyl-beta-D-glucuronide) Agar, Brain Heart Infusion (BHI), Buffered Peptone Water, were obtained from Becton Dickinson (Franklin Lakes, NJ). Tryptic Soy Agar (TSA), D-Raffinose, D-Arabinose, Bromocresol Purple, Peptone from casein, D-Xylose were obtained from Sigma-Aldrich (St. Louis, MO). D-Sorbitol was obtained from Fisher Scientific (Hampton, NH). Trehalose was obtained from GoldBio (St. Louis, MO). Bile salts was obtained from Honeywell Fluka (Charlotte, NC). CHROMagar™ STEC and CHROMagar™ SALMONELLA PLUS and were obtained from CHROMagar (Paris, France). The SSS medium, commercially known as PDX-STEC, was prepared according to instructions from the U.S. Pat. No. 9,518,283 [11], with the addition of 0.025% (m/v) bromocresol purple. The modified SSS medium or m-SSS medium was prepared by removing sulfanilamide and myricetin from the formulation. Modified tryptic soy broth (mTSB) was prepared by adding 0.15% (w/v) bile salts and 0.0008% (w/v) sodium novobiocin obtained from Sigma Aldrich, Milwaukee, WI. Modified buffered peptone water (mBPWp) was prepared according to the Bacteriological Analytical Manual of the US Food and Drug Administration [27]. Cibacron Blue 3GA was purchased from Polysciences (Warrington, PA).

Bacterial strains. STEC and Salmonella strains were obtained from the Penn State University E. coli Reference Center in University Park, Pennsylvania, the Center for Disease Control and Prevention in Atlanta, Georgia, the U.S. Meat Animal Research Center (USDA Agricultural Research Services) in Clay Center, Nebraska, the American Type Culture Collection (ATCC) in Manassas, Virginia, and the University of Minnesota Veterinary Diagnostic Laboratory in Saint Paul, Minnesota. Bacterial cultures were maintained as glycerol stock at −20° C. and revived in TSB incubated at 37° C. overnight before use.

E. coli growth stimulating activity assays. In Experiments 1 and 2 the growth effects of medium with and without putative stimulating factors were assayed by inoculating 3.0 mL of modified SSS medium or mBPWp with a STEC or Salmonella strain at ˜1 CFU/mL. After 7 h of incubation at 37° C., 0.1 mL aliquots of the samples were spread plated on CHROMAGAR™ STEC or CHROMAGAR SALMONELLA PLUS then incubated for 18 hours at 37° C. Bacterial populations were enumerated the following day by colony counts. In Experiments 3 and 4 growth stimulating assays used 1.0 mL portions of SSS medium prepared with purified components or commercial proteins, inoculated with 5-6 CFU/mL of E. coli O157:H7, that were then incubated at 37° C. for six hours, after which 0.1 mL was plated onto Chromagar STEC medium. The plates were incubated at 37° C. overnight and enumerated the following day.

Time-course experiments were conducted to monitor the growth stimulating activity on STEC and Salmonella in media with and without putative stimulating factors where 100-L aliquots were withdrawn at 3, 5, and 7 hours and plated onto CHROMAGAR™ STEC or CHROMAGAR SALMONELLA PLUS, incubated and scored as described above.

Assessing growth stimulating factor in alternate medium. Wheat kernels (25 g) were placed in stomacher bags then inoculated with ˜3 CFU of E. coli O157:H7 and held at room temperature for 20 minutes. Two hundred milliliters of mBPWp, or mBPWp supplemented with 10% (v/v) of the F-1 ammonium sulfate cut (described below) was added to the stomacher bags. The samples were enriched at 42° C. for seven hours, and 0.1-mL aliquots were taken at 2-hour intervals and spread onto CHROMAGAR™ STEC plates then incubated overnight at 37° C. Mauve colonies were enumerated.

Conditioning media. The hypothesized stimulating factor was extracted into SSS medium by incubation of ground beef in ˜3:1 v/m ratio where 165 g ground beef (80:20 lean:fat) was suspended in 500 mL SSS medium and stirred for thirty minutes at 10° C. The medium was decanted through a screen in a stomacher bag and filtered through a Celite pad to provide the conditioned medium. The conditioned medium was sterilized by filtration through a 0.22 μm filter.

Extraction procedure. Ground beef (80:20 lean:fat) was obtained from a local grocery store. Ground beef extracted was prepared by suspending 4:1 v/m in 0.02 M Tris-Cl, pH 7.9, 0.032M MgCl2, 0.027% w/v Niaproof-4. Extracts were clarified by filtration through course screen in stomacher bags followed by filtration through Celite 545 (Sigma-Aldrich, St. Louis, MO).

Ammonium sulfate precipitation and fractionation. Initial ammonium sulfate fractionation was carried out at 100% saturation to determine if the active component was salt precipitable. To 100 mL extract of the ground beef (see above), 72.9 g of solid ammonium sulfate was added with stirring at 10° C. After 30 minutes, the sample was centrifuged in a MyFuge Mini Centrifuge™, Benchmark Scientific, Edison, NJ, at 6,000 RPM to pellet the precipitate. The supernatant was collected, and the pellet was re-dissolved in 3 mL of 0.01 M Tris-Cl pH 7.8. The resuspended pellet was dialyzed against same buffer to desalt. This initial total fraction was termed “F-1”.

Ammonium sulfate fractionation was carried out by addition of solid ammonium sulfate to obtain 20% and 60% saturation. The protein precipitates obtained at all ammonium sulfate saturation levels were collected by filtration through glass fiber filters and redissolved in a minimum volume of 0.02 M Tris-Cl, pH 7.9. The ammonium sulfate fraction obtained from 20% to 60% saturation was designated “AS-20/60”.

Ammonium sulfate precipitate F-1 and fraction AS-20/60 were prepared in SSS medium to a final concentration of 10% v/v for use in Experiment 2 growth studies with STEC and Salmonella cultures (see above).

Additional Purification Procedures. The F-1 precipitate was further purified using molecular weight cut off filters as follows: The F-1 preparation was ultrafiltered using an Amicon stirred ultrafiltration cell with a 50 and 100-kD nominal molecular weight cut-off (MWCO) membranes purchased from Sterlitech (Kent, WA). The retentate and filtrate fractions were prepared at 10% v/v in SSS medium to assayed in Experiment 2 for E. coli growth stimulating activity (above).

The AS-20/60 fraction was further purified on a Cibacron Blue Sephadex column prepared according to procedures described by Turner [12], followed by polyacrylamide gel purification according to the procedure of Laemmli [13]. The AS-20/60 fraction was dialyzed into starting buffer, 0.01M MES, pH 6.1, 0.04M KCl, 0.001M dithiothreitol. Then it was loaded onto the column and washed with 5 volumes of starting buffer. The active portion was eluted from the column by washing the column with 3 volumes of starting buffer containing 0.5M NaCl. The AS-20/60 Sephadex column elutate was loaded into Mini-Protean T GX precast gels (Bio-Rad Laboratories, Hercules C A) for PAGE. Aliquots (10 to 12-uL) having 10 to 100 micrograms protein were applied to the wells and processed according to manufacturer's instructions. Preparative gels were removed from the gel forms and fixed for 15 minutes in 1M sodium acetate before negative staining using the zinc-imidazole procedure of Simpson [14]. The visualized bands were excised and minced with a razor blade. Minced bands were suspended overnight in 0.7 mL of 0.02M Tris-Cl, 0.002M dithiothreitol pH 7.9 buffer at 4° C. The supernatants obtained were prepared in mBPWp utilizing 10% v/v per assay of the material obtained from the excised protein bands for use in Experiment 3.

Tryptic digest and mass spectrometry identification of putative active components. The active fractions identified from the polyacrylamide gel purification above were excised from polyacrylamide gel slab suspended in 2.0 mL 0.02 M Tris-Cl pH 7.8. The samples were submitted to the Center for Proteomics Mass Spectroscopy facility at the University of Minnesota. The samples were digested with Trypsin and subjected to mass spectroscopic analysis according to procedures previously published [19].

Putative growth factor preparation. Rabbit muscle phosphoglucomutase (PGM) was purchased from Sigma-Aldrich (Milwaukee, WI). Keratin was purchased from Fitzgerald Industries International (Acton, MA). PGM was diluted to 1 mg/mL in 0.02 M Tris-Cl pH 7.0, 0.001 M dithiothreitol (Sigma Aldrich, Milwaukee, WI). The PGM was prepared in mBPWp at 50 μg/mL and 100 μg/mL to test growth stimulating activity. Keratin was dissolved in 0.02 M Tris-Cl pH 7.0, 0.001 M dithiothreitol at 1 mg/mL and used at 50- and 100 μg/mL in mBPWp for growth stimulating assays.

Statistical analysis. All cell count assays were performed in triplicate except where specifically mentioned above. Colony forming units (CFU) per mL were log transformed for analysis. Mean log₁₀ CFU/mL and standard deviations were calculated using the AVERAGE and SDIFF functions in MicroSoft Excel. Unpaired t-tests to identify significantly different means was performed using GraphPad Prizm quick calcs (www.graphpad.com/quickcalcs/ttest) with significant difference set at 0.05.

### Results

Experiment 1. The initial experiment used conditioned SSS medium containing ground beef extract and compared that to SSS containing traditional powdered beef extract supplement (as control) to verify STEC and Salmonella growth stimulation was directly related to the ground beef extract. The increases in 7 h populations of roughly 50-fold for E. coli O157:H7 and ˜300-fold for STEC-045 suggested a growth stimulating compound was provided by extracts from fresh ground beef, but not any compounds present in powdered beef extract (Table 1).

TABLE 1
Effect of Supplementing Media on STEC^{a} Growth^{b}

Medium Supplementation^{d}

| E. coli | | Beef Extract | Ground Beef |
|---|---|---|---|
| Strain | None | Powder^{e} | Conditioned^{f} |
| O157:H7 | <LOD^{g} | 2.1 ± 0.03 | 3.7 ± 0.024^{h} |
| STEC-O45 | <LOD | 0.4 ± 0.12 | 2.5 ± 0.018^{h} |

^{a}STEC are Shiga toxin-producing E. coli.
^{b}Values represent mean Log₁₀ CFU/mL ± standard deviation attained by a 1-3 CFU/mL of each strain following 7 h incubation at 42° C.
^{d}The highly selective SSS medium was used with supplements shown.
^{e}Beef Extract Powder was supplemented at 5% (w/v) into SSS broth.
^{f}Conditioning of media was accomplished by incubation of ground beef in SSS media (3:1 v/m ratio) stirred 30 m at 10° C., then clarified by screening and filtering before sterilized using a 0.22 μm filter.
^{g}Value below the level of detection (LOD) of 0.0 Log₁₀ CFU/mL.
^{h}The difference between the two supplements is significantly different (P < 0.05).

Experiment 2. We commenced to partially purify the putative growth stimulating factor from crude ground beef extract by ammonium sulfate precipitation. The total precipitate, referred to as F-1, was used to supplement SSS media and compared to control SSS for growth stimulating activity after 7 h of incubation. STEC (0157, 0111, and 045) at ˜1 CFU/mL were demonstrated to reach concentrations of ˜3 log CFU greater over controls that lacked the growth stimulating factor supplied by the F-1 preparation (Table 2). The results further showed that using the more concentrated F-1 preparation provided a factor of ˜100-fold (2 log) more colonies than the simply conditioned media in Experiment 1 (Table 1). Thus, demonstrating the growth stimulating factor was enriched in the F-1 preparation, and its activity was concentration dependent when examined under similar incubation conditions.

TABLE 2
Effect of Ammonium Sulfate Precipitate
Fraction 1 (F-1) on STEC^{a} growth^{b}

| | |
|---|---|
| E. coli | Medium Supplementation^{d} |

| Strain | None | F-1 Precipitate^{e} |
|---|---|---|
| O157:H7 | 2.2 ± 0.07 | >4.0^{fh} |
| STEC-O111 | 2.1 ± 0.05 | >4.0^{fh} |
| STEC-O45 | <LOD^{g} | 3.7 ± 0.03 ^{h} |

^{a}STEC are Shiga toxin-producing E. coli.
^{b}Values represent mean Log₁₀ ± standard deviation CFU/mL attained by a 1-3 CFU/mL of each strain following 7 h incubation at 42° C.
^{d}The highly selective SSS medium was used with supplements shown.
^{e}The F-1 Precipitate was a saturated ammonium sulphate precipitation from a ground beef suspension, that was used at X % (w/v) in SSS broth.
^{f}The upper limit of resolution in the colony counting assay was limited to 4.0 Log₁₀ CFU/mL with these sample results being too numerous to count.
^{g}Value below the level of detection (LOD) of 0.0 Log₁₀ CFU/mL.
^{h} The difference between the two supplements is significantly different (P < 0.05).

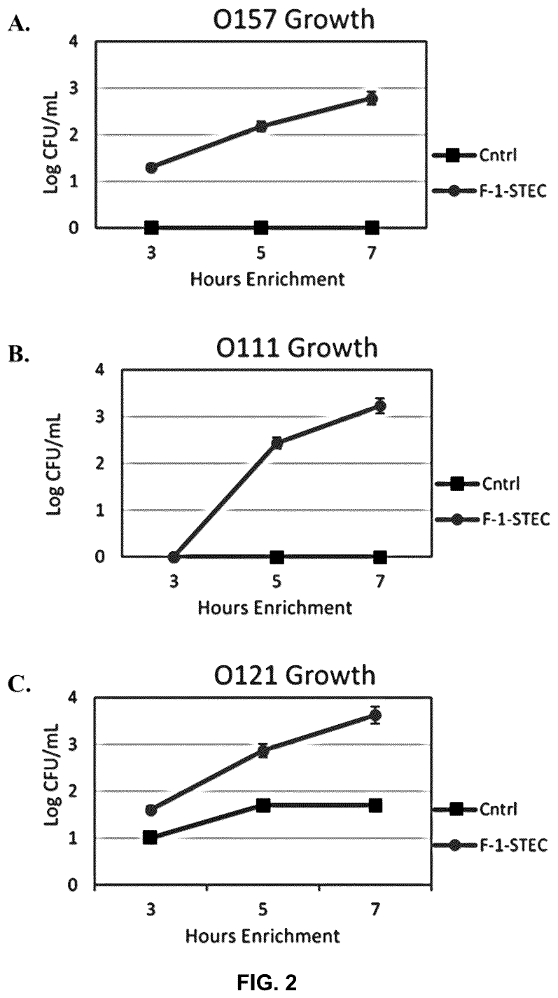

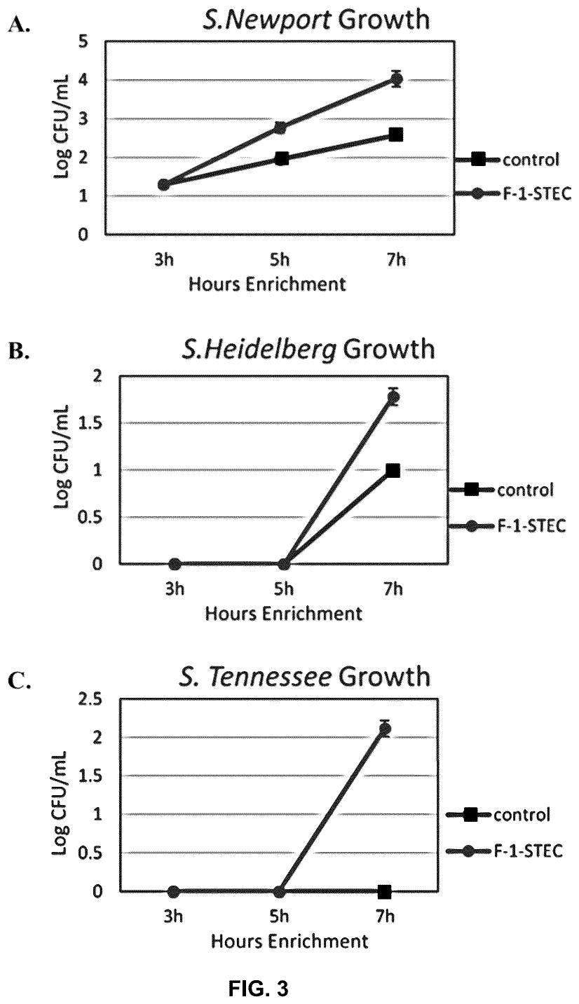

The activity of the F-1 precipitate was examined over time on STEC and Salmonella. Growth Time points were taken every two hours and compared to control cultures. Growth was observed to be accelerated for three different STEC serotypes in the presence of SSS medium supplemented with 10% v/v F-1 preparation (FIG. 1). STEC-O157:H7, —O111, and —O121 populations entered log phase growth in the presence of 10% v/v F-1 at a timepoint at where matched controls were still in lag phase growth (FIG. 2). In analogous experiments using different Salmonella serotypes (Newport, Heidelberg, and Tennessee) similar activity of 10% v/v F-1 in SSS media was observed (FIG. 3).

Having determined that the F-1 precipitate influenced STEC and Salmonella growth, the ammonium sulphate precipitation was refined by fractionation to identify where the activity was most concentrated. The precipitate formed by the 20 to 60% ammonium sulphate fraction (AS-20/60) was found to possess >90% of the stimulating activity (Table 3). Then to further characterize the growth stimulating factor, its apparent molecular weight was estimated by ultrafiltration of the AS-20/60 fraction with 50- and 100-kD nominal molecular weight cut-off (MWCO) membranes. When the filtrate and retentate of each were tested for E. coli O157:H7 growth stimulation, the 50-kD retentate and larger molecular weight fractions were found to be most active (Table 4). The molecular weight of the growth stimulating factor was considered to be >50,000 and <100,000 molecular weight for Experiment 3.

TABLE 3
Growth Stimulation of E. coli
O157:H7^{a} by Ammonium Sulfate Fractions^{b}.

| | Ammonium Sulphate Fraction^{c} | | |
|---|---|---|---|
| Control^{d} | 0-20% | 20-60% | 60-90% |
| <LOD^{e} | 0.8 ± 0.10 | 2.4 ± 0.05^{f} | <LOD |

^{a}Values represent mean Log₁₀ ± standard deviation CFU/mL attained by a 1-3 CFU/mL of E. coli O157:H7 following 7 h incubation at 42° C.
^{b}The highly selective SSS medium was used and supplemented with the ammonium sulphate fractions shown
^{c}Each fraction was used at X % (w/v) in the SSS media broth.
^{d}Control was non-supplemented SSS media.
^{e}Value below the level of detection (LOD) of 0.0 Log₁₀ CFU/mL.
^{f}The difference between the values for this ammonium sulphate fraction compared to the control is significantly different (P < 0.05).

TABLE 4
Growth Stimulation of E. coli
O157:H7^{a} by ultrafiltration fractions of AS-20/60^{b}.

| | 50 kD MWCO^{c} | | 100 kD MWCO | |
|---|---|---|---|---|
| Control^{d} | Filtrate^{e} | Retentate^{f} | Filtrate | Retentate |
| <LOD^{g} | <LOD | 4.2 ± 0.06^{h} | 4.2 ± 0.04^{h} | 4.3 ± 0.02^{h} |

^{a}Values represent mean Log₁₀ ± standard deviation CFU/mL attained by a 1-3 CFU/mL of E. coli O157:H7 following 7 h incubation at 42° C.
^{b}The highly selective SSS medium was used and supplemented with the ultrafiltrate fractions shown. Each fraction was used at X % (v/v) in the SSS media broth.
^{c}MWCO = Molecular Weight Cut Off.
^{d}Control was non-supplemented SSS media.
^{e}Filtrate was the fraction that passed through the MWCO membrane, and is presumed to contain proteins lower than the MWCO.
^{f}Retentate was the fraction that did not pass through the MWCO, and is presumed to contain proteins greater than the MWCO.
^{g}Value below the level of detection (LOD) of 0.0 Log₁₀ CFU/mL.
^{h}The difference between the value for this filtrate or retentate compared to the control is significantly different (P < 0.05).

Experiment 3. Candidate compounds responsible for growth stimulation were identified and characterized. To obtain higher purity preparation than ammonium sulfate fractionation we subjected the AS-20/60 fraction to purification on CibaCron Blue Sephadex that efficiently bound the growth stimulating substance. The growth stimulating activity was eluted from the affinity matrix and was further resolved by SDS PAGE. This resulted in several SDS PAGE bands predominantly in the 30- to 60-kD range with the most intensely staining bands at approximately 35-, 42-, and 52-kD. Similar suspect molecular weight proteins were observed on negatively stained non-denaturing PAGE. PAGE bands of approximately 20-, 35-, 42- and 52-kD were excised and tested for E. coli O157:H7 growth stimulating activity in mBPWp (Table 5). PAGE bands of approximately 20, 35, and 52-kD increased E. coli O157:H7 growth by 0.5 to 0.8 Log₁₀, while the prominent 42 kD band had no activity. The bands corresponding to the 35-, 42-, and 52-kD proteins were submitted for mass spectroscopic analysis of their tryptic digests.

TABLE 5
Growth Stimulation of E. coli
O157:H7^{a} by PAGE Gel Band Preparations^{b}.

| | PAGE Gel Band Preparation^{c} | | | |
|---|---|---|---|---|
| Control^{d} | 20 kD | 35 kD | 42 kD | 52 kD |
| 2.7 ± 0.01 | 3.2 ± 0.01^{e} | 3.1 ± 0.01^{e} | 2.6 ± 0.04^{e} | 3.3 ± 0.04^{e} |

^{a}Values represent mean Log₁₀ ± standard deviation CFU/mL attained by a 1-3 CFU/mL of E. coli O157:H7 following 7 h incubation at 42° C.
^{b}Prominent non-denaturing PAGE gel bands were excised, minced and extracted to determine which possessed growth stimulating activity.
^{c}Each preparation was used at 10% (v/v) in mBPWp broth.
^{d}Control was non-supplemented mBPWp.
^{e}The difference between the value for this band preparation compared to the control is significantly different (P < 0.05).

The resulting mass spectroscopy data (Tables 6, 7, and 8) showed a large percentage of the spectrum in the 35-kD protein band corresponded to keratin type proteins, KRT 2, KRT10, KRT 14 and KRT 9; whereas the mass spectrum data from the excised 52-kD band was mostly devoid of any keratin proteins. The one protein appearing in the MS analysis of both the 35-kD and 52-kD protein bands was phosphoglucomutase. The spectrum of the 42-kD protein, the inactive band, was primarily creatine phosphokinase.

TABLE 6
Scaffold file of mass spectrum of 52-kD excised protein band.

| | | | | Percentage |
|---|---|---|---|---|
| | | | Molecular | Total |
| # | Identified Proteins (61) | Alternate ID | Weight | Spectrum |
| 1 | Serum albumin OS = Bos taurus | ALB | 69 kDa | 4.90% |
| | OX = 9913 GN = ALB | | | |
| | PE = 4 SV = 1 | | | |
| 2 | Methanethiol oxidase OS = Bos taurus | SELENBP1 | 53 kDa | 2.00% |
| | OX = 9913 GN = SELENBP1 | | | |
| | PE = 1 SV = 1 | | | |
| 3 | Glucose-6-phosphate isomerase | GPI | 64 kDa | 0.99% |
| | OS = Bos taurus OX = 9913 | | | |
| | GN = GPI PE = 1 SV = 1 | | | |
| 4 | Retinal dehydrogenase 1 | ALDH1A1 | 55 kDa | 0.82% |
| | OS = Bos taurus OX = 9913 | | | |
| | GN = ALDH1A1 PE = 1 SV = 3 | | | |
| 5 | Aldehyde dehydrogenase, mitochondrial | ALDH2 | 57 kDa | 0.66% |
| | OS = Bos taurus OX = 9913 | | | |
| | GN = ALDH2 PE = 1 SV = 2 | | | |
| 6 | Trypsin OS = Sus scrofa OX = 9823 | | 24 kDa | 0.58% |
| | PE = 1 SV = 1 | | | |
| 7 | Thioredoxin reductase 1, cytoplasmic | TXNRD1 | 55 kDa | 0.39% |
| | OS = Bos taurus OX = 9913 | | | |
| | GN = TXNRD1 PE = 2 SV = 3 | | | |
| 8 | Keratin, type II cytoskeletal 1 | KRT1 | 66 kDa | 0.37% |
| | OS = Homo sapiens OX = 9606 | | | |
| | GN = KRT1 PE = 1 SV = 6 | | | |
| 9 | PGM5 protein OS = Bos taurus | PGM5 | 62 kDa | 0.33% |
| | OX =9913 GN = PGM5 | | | |
| | PE = 2 SV = 1 | | | |
| 10 | Glutathione S-transferase P | GSTP1 | 24 kDa | 0.41% |
| | OS = Bos taurus OX = 9913 | | | |
| | GN = GSTP1 PE = 1 SV = 2 | | | |
| 11 | Alpha-1-antiproteinase OS = Bos taurus | SERPINA1 | 46 kDa | 0.37% |
| | OX = 9913 GN = SERPINA1 | | | |
| | PE = 1 SV = 1 | | | |
| 12 | Cytosol aminopeptidase OS = Bos taurus | LAP3 | 56 kDa | 0.33% |
| | OX = 9913 GN = LAP3 | | | |
| | PE = 1 SV = 3 | | | |
| 13 | Cluster of Keratin, type I cytoskeletal 10 | KRT10 | 59 kDa | 0.35% |
| | OS = Homo sapiens OX = 9606 | | | |
| | GN = KRT10 PE = 1 SV = 6 | | | |
| | (P13645) | | | |
| 13.1 | Keratin, type I cytoskeletal 10 | KRT10 | 59 kDa | 0.27% |
| | OS = Homo sapiens OX = 9606 | | | |
| | GN = KRT10 PE = 1 SV = 6 | | | |
| 13.2 | Keratin, type I cytoskeletal 14 | KRT14 | 50 kDa | 0.12% |
| | OS = Bos taurus OX = 9913 | | | |
| | GN = KRT14 PE = 1 SV = 1 | | | |
| 13.3 | IF rod domain-containing protein | KRT13 | 47 kDa | 0.10% |
| | OS = Bos taurus OX = 9913 | | | |
| | GN = KRT13 PE = 3 SV = 1 | | | |
| 13.4 | Keratin, type I cytoskeletal 19 | KRT19 | 44 kDa | 0 |
| | OS = Bos taurus OX = 9913 | | | |
| | GN = KRT19 PE = 2 SV = 1 | | | |
| 14 | Hemopexin OS = Bos taurus | HPX | 52 kDa | 0.35% |
| | OX = 9913 GN = HPX | | | |
| | PE = 2 SV = 1 | | | |
| 15 | Keratin, type I cytoskeletal 9 | KRT9 | 62 kDa | 0.27% |
| | OS = Homo sapiens OX = 9606 | | | |
| | GN = KRT9 PE = 1 SV = 3 | | | |
| 16 | Uncharacterized protein OS = Bos taurus | | 35 kDa | 0.27% |
| | OX = 9913 PE = 1 SV = 1 | | | |
| 17 | Alanine aminotransferase 1 | GPT | 88 kDa | 0.25% |
| | OS = Bos taurus OX = 9913 | | | |
| | GN = GPT PE = 4 SV = 1 | | | |
| 18 | Dihydrolipoyl dehydrogenase | DLD | 54 kDa | 0.25% |
| | OS = Bos taurus OX = 9913 | | | |
| | GN = DLD PE = 1 SV = 2 | | | |
| 19 | Keratin, type II cytoskeletal 2 epidermal | KRT2 | 65 kDa | 0.23% |
| | OS = Homo sapiens OX = 9606 | | | |
| | GN = KRT2 PE = 1 SV = 2 | | | |
| 20 | Creatine kinase B-type | CKB | 43 kDa | 0.25% |
| | OS = Bos taurus OX = 9913 | | | |
| | GN = CKB PE = 1 SV = 1 | | | |
| 21 | Glutathione reductase | GSR | 56 kDa | 0.23% |
| | OS = Bos taurus OX = 9913 | | | |
| | GN = GSR PE = 3 SV = 3 | | | |
| 22 | Rab GDP dissociation inhibitor alpha | GDI1 | 51 kDa | 0.21% |
| | OS = Bos taurus OX = 9913 | | | |
| | GN = GDI1 PE = 1 SV = 1 | | | |
| 23 | Serotransferrin | TF | 78 kDa | 0.23% |
| | OS = Bos taurus OX = 9913 | | | |
| | GN = TF PE = 1 SV = 2 | | | |
| 24 | WD repeat-containing protein 1 | WDR1 | 66 kDa | 0.18% |
| | OS = Bos taurus OX = 9913 | | | |
| | GN = WDR1 PE = 4 SV = 2 | | | |
| 25 | Phosphoglucomutase-1 | PGM1 | 62 kDa | 0.16% |
| | OS = Bos taurus OX = 9913 | | | |
| | GN = PGM1 PE = 3 SV = 1 | | | |
| 26 | Lymphocyte cytosolic protein 1 | LCP1 | 70 kDa | 0.19% |
| | OS = Bos taurus OX = 9913 | | | |
| | GN = LCP1 PE = 1 SV = 1 | | | |
| 27 | Peptidase D OS = Bos taurus | PEPD | 55 kDa | 0.18% |
| | OX = 9913 GN = PEPD | | | |
| | PE = 3 SV = 1 | | | |
| 28 | Phosphoglucomutase 2 OS = Bos taurus | PGM2 | 67 kDa | 0.16% |
| | OX = 9913 GN = PGM2 | | | |
| | PE = 1 SV = 1 | | | |
| 29 | Thioredoxin reductase 2, mitochondrial | TXNRD2 | 55 kDa | 0.18% |
| | OS = Bos taurus OX = 9913 | | | |
| | GN = TXNRD2 PE = 1 SV = 2 | | | |
| 30 | Serpin A3-3 OS = Bos taurus | SERPINA3-3 | 46 kDa | 0.12% |
| | OX = 9913 GN = SERPINA3-3 | | | |
| | PE = 1 SV = 1 | | | |
| 31 | Hydroxyacyl-CoA dehydrogenase | HADH | 34 kDa | 0.14% |
| | OS = Bos taurus OX = 9913 | | | |
| | GN = HADH PE = 4 SV = 1 | | | |
| 32 | Carboxypeptidase B2 OS = Bos taurus | CPB2 | 44 kDa | 0.08% |
| | OX = 9913 GN = CPB2 | | | |
| | PE = 4 SV = 1 | | | |
| 33 | Glucosylceramidase beta 3 | GBA3 | 54 kDa | 0.08% |
| | OS = Bos taurus OX = 9913 | | | |
| | GN = GBA3 PE = 3 SV = 3 | | | |
| 34 | Cluster of SERPIN domain-containing protein | LOC112445741 | 45 kDa | 0.08% |
| | OS = Bos taurus OX = 9913 | | | |
| | GN = LOC112445741 PE = 3 SV = 1 | | | |
| | (A0A3Q1MGZ6) | | | |
| 34.1 | SERPIN domain-containing protein | LOC112445741 | 45 kDa | 0.06% |
| | OS = Bos taurus OX = 9913 | | | |
| | GN = LOC112445741 PE = 3 SV = 1 | | | |
| 34.2 | Serpin A3-7 OS = Bos taurus | SERPINA3-7 | 47 kDa | 0.06% |
| | OX = 9913 GN = SERPINA3-7 | | | |
| | PE = 1 SV = 1 | | | |
| 35 | Alpha-enolase OS = Bos taurus | ENO1 | 45 kDa | 0.08% |
| | OX = 9913 GN = ENO1 | | | |
| | PE = 3 SV = 1 | | | |
| 36 | Inter-alpha-trypsin inhibitor heavy chain H4 | ITIH4 | 101 kDa | 0.08% |
| | OS = Bos taurus OX = 9913 | | | |
| | GN = ITIH4 PE = 4 SV = 1 | | | |
| 37 | Aldo-keto reductase family 1 member A1 | AKR1A1 | 37 kDa | 0.08% |
| | OS = Bos taurus OX = 9913 | | | |
| | GN = AKR1A1 PE = 2 SV = 1 | | | |
| 38 | Bifunctional purine biosynthesis protein ATIC | ATIC | 64 kDa | 0.06% |
| | OS = Bos taurus OX = 9913 | | | |
| | GN = ATIC PE = 2 SV = 1 | | | |
| 39 | Uncharacterized protein | | 40 kDa | 0.10% |
| | OS = Bos taurus OX = 9913 | | | |
| | PE = 1 SV = 1 | | | |
| 40 | Aldo-keto reductase family 1 member B1 | AKR1B1 | 36 kDa | 0.06% |
| | OS = Bos taurus OX = 9913 | | | |
| | GN = AKR1B1 PE = 1 SV = 2 | | | |
| 41 | Ceruloplasmin OS = Bos taurus | CP | 116 kDa | 0.06% |
| | OX = 9913 GN = CP | | | |
| | PE = 1 SV = 1 | | | |
| 42 | Superoxide dismutase [Cu—Zn] | SOD3 | 27 kDa | 0.04% |
| | OS = Bos taurus OX = 9913 | | | |
| | GN = SOD3 PE = 1 SV = 1 | | | |
| 43 | Alpha-1B-glycoprotein | A1BG | 62 kDa | 0.04% |
| | OS = Bos taurus OX = 9913 | | | |
| | GN = A1BG PE = 1 SV = 1 | | | |
| 44 | Transthyretin OS = Bos taurus | TTR | 20 kDa | 0.04% |
| | OX = 9913 GN = TTR | | | |
| | PE = 3 SV = 1 | | | |
| 45 | Cluster of SERPIN domain-containing protein | LOC511695 | 45 kDa | 0.04% |
| | OS = Bos taurus OX = 9913 | | | |
| | GN = LOC511695 PE = 3 SV = 1 | | | |
| | (A0A3Q1LY36) | | | |
| 45.1 | SERPIN domain-containing protein | LOC511695 | 45 kDa | 0.04% |
| | OS = Bos taurus OX = 9913 | | | |
| | GN = LOC511695 PE = 3 SV = 1 | | | |
| 45.2 | SERPIN domain-containing protein | LOC511106 | 44 kDa | 0.02% |
| | OS = Bos taurus OX = 9913 | | | |
| | GN = LOC511106 PE = 3 SV = 3 | | | |
| 46 | Alpha-aminoadipic semialdehyde dehydrogenase | ALDH7A1 | 59 kDa | 0.04% |
| | OS = Bos taurus OX = 9913 | | | |
| | GN = ALDH7A1 PE = 3 SV = 1 | | | |
| 47 | Cluster of Glutathione S-transferase | GSTM3 | 28 kDa | 0.04% |
| | OS = Bos taurus OX = 9913 | | | |
| | GN = GSTM3 PE = 1 SV = 1 | | | |
| | (A0A3Q1LSN6) | | | |
| 47.1 | Glutathione S-transferase | GSTM3 | 28 kDa | 0.04% |
| | OS = Bos taurus OX = 9913 | | | |
| | GN = GSTM3 PE = 1 SV = 1 | | | |
| 47.2 | Glutathione S-transferase | GSTM2 | 26 kDa | 0 |
| | OS = Bos taurus OX = 9913 | | | |
| | GN = GSTM2 PE = 3 SV = 1 | | | |
| 48 | Transgelin OS = Bos taurus | TAGLN | 23 kDa | 0.04% |
| | OX = 9913 GN = TAGLN | | | |
| | PE = 1 SV = 4 | | | |
| 49 | SERPIN domain-containing protein | LOC112445470 | 28 kDa | 0.04% |
| | OS = Bos taurus OX = 9913 | | | |
| | GN = LOC112445470 PE = 3 SV = 1 | | | |
| 50 | EMAP like 2 OS = Bos taurus | EML2 | 98 kDa | 0.04% |
| | OX = 9913 GN = EML2 | | | |
| | PE = 4 SV = 1 | | | |
| 51 | Fascin OS = Bos taurus OX = 9913 | FSCN1 | 55 kDa | 0.04% |
| | GN = FSCN1 PE = 1 SV = 1 | | | |
| 52 | KRT5 protein OS = Bos taurus | KRT5 | 63 kDa | 0.06% |
| | OX = 9913 GN = KRT5 | | | |
| | PE = 2 SV = 1 | | | |
| 53 | Fructose-bisphosphate aldolase | ALDOA | 39 kDa | 0.04% |
| | OS = Bos taurus OX = 9913 | | | |
| | GN = ALDOA PE = 1 SV = 1 | | | |
| 54 | Keratin, type II cytoskeletal 79 | KRT79 | 57 kDa | 0.04% |
| | OS = Bos taurus OX = 9913 | | | |
| | GN = KRT79 PE = 3 SV = 1 | | | |
| 55 | IF rod domain-containing protein | KRT6A | 61 kDa | 0.04% |
| | OS = Bos taurus OX = 9913 | | | |
| | GN = KRT6A PE = 3 SV = 1 | | | |

TABLE 7
Scaffold file of mass spectrum of 35-kD excised protein band.

| | | | | Percentage |
|---|---|---|---|---|
| | | | Molecular | of Total |
| # | Identified Proteins (55) | Alternate ID | Weight | Spectrum |
| 1 | Cluster of Keratin, type II | KRT2 | 65 kDa | 2.90% |
| | cytoskeletal 2 epidermal | | | |
| | OS = Homo sapiens OX = 9606 | | | |
| | GN = KRT2 PE = 1 SV = 2 | | | |
| | (P35908) | | | |
| 1.1 | Keratin, type II cytoskeletal 2 epidermal | KRT2 | 65 kDa | 1.70% |
| | OS = Homo sapiens OX = 9606 | | | |
| | GN = KRT2 PE = 1 SV = 2 | | | |
| 1.2 | KRT5 protein OS = Bos taurus | KRT5 | 63 kDa | 0.67% |
| | OX = 9913 GN = KRT5 | | | |
| | PE = 2 SV = 1 | | | |
| 1.3 | Keratin 3 OS = Bos taurus | KRT3 | 64 kDa | 0.64% |
| | OX = 9913 GN = KRT3 | | | |
| | PE = 1 SV = 1 | | | |
| 1.4 | IF rod domain-containing protein | KRT6A | 61 kDa | 0.47% |
| | OS = Bos taurus OX = 9913 | | | |
| | GN = KRT6A PE = 3 SV = 1 | | | |
| 1.5 | KRT4 protein OS = Bos taurus | KRT4 | 58 kDa | 0.27% |
| | OX = 9913 GN = KRT4 | | | |
| | PE = 2 SV = 1 | | | |
| 1.6 | Keratin, type II cytoskeletal 75 | KRT75 | 59 kDa | 0.27% |
| | OS = Bos taurus OX = 9913 | | | |
| | GN = KRT75 PE = 2 SV = 1 | | | |
| 1.7 | Keratin 77 OS = Bos taurus | KRT77 | 63 kDa | 0.24% |
| | OX = 9913 GN = KRT77 | | | |
| | PE = 1 SV = 1 | | | |
| 1.8 | Keratin, type II cytoskeletal 79 | KRT79 | 57 kDa | 0.17% |
| | OS = Bos taurus OX = 9913 | | | |
| | GN = KRT79 PE = 3 SV = 1 | | | |
| 2 | Cluster of Keratin, type I cytoskeletal 10 | KRT10 | 59 kDa | 2.60% |
| | OS = Homo sapiens OX = 9606 | | | |
| | GN = KRT10 PE = 1 SV = 6 | | | |
| | (P13645) | | | |
| 2.1 | Keratin, type I cytoskeletal 10 | KRT10 | 59 kDa | 2.60% |
| | OS = Homo sapiens OX = 9606 | | | |
| | GN = KRT10 PE = 1 SV = 6 | | | |
| 2.2 | Keratin, type I cytoskeletal 15 | KRT15 | 49 kDa | 0.44% |
| | OS = Ovis aries OX = 9940 | | | |
| | GN = KRT15 PE = 2 SV = 1 | | | |
| 3 | Keratin, type II cytoskeletal 1 | KRT1 | 66 kDa | 2.30% |
| | OS = Homo sapiens OX = 9606 | | | |
| | GN = KRT1 PE = 1 SV = 6 | | | |
| 4 | Carbonic anhydrase 3 | CA3 | 29 kDa | 1.40% |
| | OS = Bos taurus OX = 9913 | | | |
| | GN = CA3 PE = 2 SV = 3 | | | |
| 5 | Cluster of Keratin, type I cytoskeletal 14 | KRT14 | 56 kDa | 1.20% |
| | OS = Bos taurus OX = 9913 | | | |
| | GN = KRT14 PE = 1 SV = 3 | | | |
| | (F1MC11) | | | |
| 5.1 | Keratin, type I cytoskeletal 14 | KRT14 | 56 kDa | 1.10% |
| | OS = Bos taurus OX = 9913 | | | |
| | GN = KRT14 PE = 1 SV = 3 | | | |
| 5.2 | Keratin, type I cytoskeletal 17 | KRT17 | 49 kDa | 0.71% |
| | OS = Bos taurus OX = 9913 | | | |
| | GN = KRT17 PE = 3 SV = 1 | | | |
| 5.3 | Keratin 42 OS = Bos taurus | KRT42 | 50 kDa | 0.54% |
| | OX = 9913 GN = KRT42 | | | |
| | PE = 1 SV = 1 | | | |
| 6 | Keratin, type I cytoskeletal 9 | KRT9 | 62 kDa | 0.98% |
| | OS = Homo sapiens OX = 9606 | | | |
| | GN = KRT9 PE = 1 SV = 3 | | | |
| 7 | Phosphoglucomutase-1 OS = Bos taurus | PGM1 | 62 kDa | 0.91% |
| | OX = 9913 GN = PGM1 | | | |
| | PE = 3 SV = 1 | | | |
| 8 | Trypsin OS = Sus scrofa OX = 9823 PE = 1 SV = 1 | | 24 kDa | 0.81% |
| 9 | Fructose-bisphosphate aldolase | ALDOA | 39 kDa | 0.54% |
| | OS = Bos taurus OX = 9913 | | | |
| | GN = ALDOA PE = 1 SV = 1 | | | |
| 10 | Desmoplakin OS = Bos taurus | DSP | 332 kDa | 0.57% |
| | OX = 9913 GN = DSP | | | |
| | PE = 1 SV = 2 | | | |
| 11 | Pyridoxal phosphate homeostasis protein | PLPBP | 30 kDa | 0.44% |
| | OS = Bos taurus OX = 9913 | | | |
| | GN = PLPBP PE = 2 SV = 1 | | | |
| 12 | L-lactate dehydrogenase A chain | LDHA | 37 kDa | 0.44% |
| | OS = Bos taurus OX = 9913 | | | |
| | GN = LDHA PE = 2 SV = 2 | | | |
| 13 | Glutathione S-transferase A4 | GSTA4 | 26 kDa | 0.34% |
| | OS = Bos taurus OX = 9913 | | | |
| | GN = GSTA4 PE = 2 SV = 1 | | | |
| 14 | Flavin reductase (NADPH) | BLVRB | 21 kDa | 0.30% |
| | OS = Bos taurus OX = 9913 | | | |
| | GN = BLVRB PE = 1 SV = 1 | | | |
| 15 | Junction plakoglobin | JUP | 81 kDa | 0.34% |
| | OS = Bos taurus OX = 9913 | | | |
| | GN = JUP PE = 1 SV = 1 | | | |
| 16 | NAD(P)H dehydrogenase, quinone 1 | NQO1 | 31 kDa | 0.24% |
| | OS = Bos taurus OX = 9913 | | | |
| | GN = NQO1 PE = 2 SV = 1 | | | |
| 17 | Desmoglein-1 OS = Bos taurus | DSG1 | 112 kDa | 0.24% |
| | OX = 9913 GN = DSG1 | | | |
| | PE = 4 SV = 2 | | | |
| 18 | Hemoglobin subunit beta | HBB | 16 kDa | 0.13% |
| | OS = Bos taurus OX = 9913 | | | |
| | GN = HBB PE = 1 SV = 1 | | | |
| 19 | GTP:AMP phosphotransferase AK3, | AK3 | 26 kDa | 0.17% |
| | mitochondrial OS = Bos taurus | | | |
| | OX = 9913 GN = AK3 | | | |
| | PE = 1 SV = 3 | | | |
| 20 | Carboxymethylenebutenolidase homolog | CMBL | 28 kDa | 0.17% |
| | OS = Bos taurus OX = 9913 | | | |
| | GN = CMBL PE = 4 SV = 1 | | | |
| 21 | Carbonic anhydrase OS = Bos taurus | CA2 | 29 kDa | 0.13% |
| | OX = 9913 GN = CA2 | | | |
| | PE = 1 SV = 3 | | | |
| 22 | Uncharacterized protein | GSTM2 | 26 kDa | 0.20% |
| | OS = Bos taurus OX = 9913 | | | |
| | GN = GSTM2 PE = 4 SV = 2 | | | |
| 23 | Pyruvate kinase OS = Bos taurus | PKM | 58 kDa | 0.13% |
| | OX = 9913 GN = PKM | | | |
| | PE = 1 SV = 1 | | | |
| 24 | Keratin 24 OS = Bos taurus | KRT24 | 55 kDa | 0.17% |
| | OX = 9913 GN = KRT24 | | | |
| | PE = 3 SV = 3 | | | |
| 25 | Annexin A2 OS = Bos taurus | ANXA2 | 39 kDa | 0.10% |
| | OX = 9913 GN = ANXA2 | | | |
| | PE = 1 SV = 2 | | | |
| 26 | Peroxiredoxin-1 OS = Bos taurus | PRDX1 | 22 kDa | 0.10% |
| | OX = 9913 GN = PRDX1 | | | |
| | PE = 2 SV = 1 | | | |
| 27 | Hydroxyacylglutathione hydrolase, | HAGH | 61 kDa | 0.10% |
| | mitochondrial OS = Bos taurus | | | |
| | OX = 9913 GN = HAGH | | | |
| | PE = 1 SV = 1 | | | |
| 28 | Triosephosphate isomerase | TPI1 | 31 kDa | 0.10% |
| | OS = Bos taurus OX = 9913 | | | |
| | GN = TPI1 PE = 3 SV = 1 | | | |
| 29 | Glutathione S-transferase | GSTM3 | 28 kDa | 0.13% |
| | OS = Bos taurus OX = 9913 | | | |
| | GN = GSTM3 PE = 1 SV = 1 | | | |
| 30 | Glycerol-3-phosphate dehydrogenase | GPD1 | 49 kDa | 0.10% |
| | [NAD(+)] OS = Bos taurus | | | |
| | OX = 9913 GN = GPD1 | | | |
| | PE = 3 SV = 1 | | | |
| 31 | Uncharacterized protein | LOC100847119 | 25 kDa | 0.07% |
| | OS = Bos taurus OX = 9913 | | | |
| | GN = LOC100847119 PE = 1 SV = 2 | | | |
| 32 | Serum albumin OS = Bos taurus | ALB | 69 kDa | 0.20% |
| | OX = 9913 GN = ALB | | | |
| | PE = 4 SV = 1 | | | |
| 33 | Glyceraldehyde-3-phosphate dehydrogenase | GAPDH | 41 kDa | 0.10% |
| | OS = Bos taurus OX = 9913 | | | |
| | GN = GAPDH PE = 1 SV = 1 | | | |
| 34 | Plakophilin-1 OS = Bos taurus | PKP1 | 80 kDa | 0.07% |
| | OX = 9913 GN = PKP1 | | | |
| | PE = 4 SV = 2 | | | |
| 35 | GLOBIN domain-containing protein | HBA1 | 14 kDa | 0.07% |
| | OS = Bos taurus OX = 9913 | | | |
| | GN = HBA1 PE = 3 SV = 1 | | | |
| 36 | Peroxiredoxin-2 OS = Bos taurus | PRDX2 | 22 kDa | 0.07% |
| | OX = 9913 GN = PRDX2 | | | |
| | PE = 2 SV = 1 | | | |
| 37 | Four and a half LIM domains 1 | FHL 1 | 36 kDa | 0.07% |
| | OS = Bos taurus OX = 9913 | | | |
| | GN = FHL1 PE = 1 SV = 2 | | | |
| 38 | LIM domain binding 3 | LDB3 | 66 kDa | 0.13% |
| | OS = Bos taurus OX = 9913 | | | |
| | GN = LDB3 PE = 4 SV = 1 | | | |
| 39 | GTP-binding protein SAR1b | SAR1B | 22 kDa | 0.07% |
| | OS = Bos taurus OX = 9913 | | | |
| | GN = SAR1B PE = 2 SV = 1 | | | |
| 40 | Creatine kinase M-type | CKM | 43 kDa | 0.07% |
| | OS = Bos taurus OX = 9913 | | | |
| | GN = CKM PE = 1 SV = 2 | | | |
| 41 | Hydroxyacyl-CoA dehydrogenase | HADH | 34 kDa | 0.07% |
| | OS = Bos taurus OX = 9913 | | | |
| | GN = HADH PE = 4 SV = 1 | | | |
| 42 | Actin, cytoplasmic 1 OS = Bos taurus | ACTB | 42 kDa | 0.10% |
| | OX = 9913 GN = ACTB | | | |
| | PE = 1 SV = 1 | | | |
| 43 | Aconitate hydratase, mitochondrial | ACO2 | 85 kDa | 0.07% |
| | OS = Bos taurus OX = 9913 | | | |
| | GN = ACO2 PE = 1 SV = 1 | | | |
| 44 | Filamin A interacting protein 1 like | FILIPIL | 135 kDa | 0.07% |
| | OS = Bos taurus OX = 9913 | | | |
| | GN = FILIP1L PE = 4 SV = 3 | | | |
| 45 | Keratin, type II cytoskeletal 80 | KRT80 | 49 kDa | 0.07% |
| | OS = Bos taurus OX = 9913 | | | |
| | GN = KRT80 PE = 3 SV = 1 | | | |

TABLE 8
Scaffold file of mass spectrum of 42-kD excised protein band.

| | | | | Percentage |
|---|---|---|---|---|
| | | | Molecular | of Total |
| # | Identified Proteins (31) | Alternate ID | Weight | Spectrum |
| 1 | Creatine kinase B-type | CKB | 43 kDa | 4.20% |
| | OS = Bos taurus OX = 9913 | | | |
| | GN = CKB PE = 1 SV = 1 | | | |
| 2 | Serum albumin OS = Bos taurus | ALB | 69 kDa | 0.45% |
| | OX = 9913 GN = ALB | | | |
| | PE = 4 SV = 1 | | | |
| 3 | Cluster of Keratin, type II cytoskeletal 1 | KRT1 | 66 kDa | 0.56% |
| | OS = Homo sapiens OX = 9606 | | | |
| | GN = KRT1 PE = 1 SV = 6 | | | |
| | (P04264) | | | |
| 3.1 | Keratin, type II cytoskeletal 1 | KRT1 | 66 kDa | 0.49% |
| | OS = Homo sapiens OX = 9606 | | | |
| | GN = KRT1 PE = 1 SV = 6 | | | |
| 3.2 | Keratin 1 OS = Bos taurus | KRT1 | 63 kDa | 0.15% |
| | OX = 9913 GN = KRT1 | | | |
| | PE = 1 SV = 2 | | | |
| 4 | Cluster of Keratin, type I cytoskeletal 10 | KRT10 | 59 kDa | 0.37% |
| | OS = Homo sapiens OX = 9606 | | | |
| | GN = KRT10 PE = 1 SV = 6 | | | |
| | (P13645) | | | |
| 4.1 | Keratin, type I cytoskeletal 10 | KRT10 | 59 kDa | 0.37% |
| | OS = Homo sapiens OX = 9606 | | | |
| | GN = KRT10 PE = 1 SV = 6 | | | |
| 4.2 | Keratin, type I cytoskeletal 14 | KRT14 | 50 kDa | 0.15% |
| | OS = Bos taurus OX = 9913 | | | |
| | GN = KRT14 PE = 1 SV = 1 | | | |
| 4.3 | Keratin, type I cytoskeletal 25 | KRT25 | 49 kDa | 0.08% |
| | OS = Bos taurus OX = 9913 | | | |
| | GN = KRT25 PE = 2 SV = 1 | | | |
| 5 | Keratin, type I cytoskeletal 9 | KRT9 | 62 kDa | 0.37% |
| | OS = Homo sapiens OX = 9606 | | | |
| | GN = KRT9 PE = 1 SV = 3 | | | |
| 6 | Acetyl-CoA acetyltransferase, mitochondrial | ACAT1 | 45 kDa | 0.37% |
| | OS = Bos taurus OX = 9913 | | | |
| | GN = ACAT1 PE = 2 SV = 1 | | | |
| 7 | Aspartate aminotransferase, cytoplasmic | GOT1 | 46 kDa | 0.30% |
| | OS = Bos taurus OX = 9913 | | | |
| | GN = GOT1 PE = 1 SV = 3 | | | |
| 8 | Fumarylacetoacetase OS = Bos taurus | FAH | 45 kDa | 0.22% |
| | OX = 9913 GN = FAH | | | |
| | PE = 3 SV = 1 | | | |
| 9.1 | Keratin, type II cytoskeletal 2 epidermal | KRT2 | 65 kDa | 0.22% |
| | OS = Homo sapiens OX = 9606 | | | |
| | GN = KRT2 PE = 1 SV = 2 | | | |
| 9.2 | IF rod domain-containing protein | KRT6A | 61 kDa | 0.08% |
| | OS = Bos taurus OX = 9913 | | | |
| | GN = KRT6A PE = 3 SV = 1 | | | |
| 9.3 | Keratin 3 OS = Bos taurus | KRT3 | 64 kDa | 0.04% |
| | OX = 9913 GN = KRT3 | | | |
| | PE = 1 SV = 1 | | | |
| 10 | Fructose-bisphosphate aldolase | ALDOA | 39 kDa | 0.15% |
| | OS = Bos taurus OX = 9913 | | | |
| | GN = ALDOA PE = 1 SV = 1 | | | |
| 11 | Trypsin OS = Sus scrofa | | 24 kDa | 0.19% |
| | OX = 9823 PE = 1 SV = 1 | | | |
| 12 | Vinculin OS = Bos taurus | VCL | 111 kDa | 0.11% |
| | OX = 9913 GN = VCL | | | |
| | PE = 1 SV = 1 | | | |
| 13 | Cluster of SERPIN domain-containing protein | | 45 kDa | 0.19% |
| | OS = Bos taurus OX = 9913 | | | |
| | PE = 3 SV = 2 (F1MMS7) | | | |
| 13.1 | SERPIN domain-containing protein | | 45 kDa | 0.15% |
| | OS = Bos taurus OX = 9913 | | | |
| | PE = 3 SV = 2 | | | |
| 13.2 | SERPIN domain-containing protein | LOC511106 | 44 kDa | 0.15% |
| | OS = Bos taurus OX = 9913 | | | |
| | GN = LOC511106 PE = 3 SV = 3 | | | |
| 14 | Creatine kinase M-type | CKM | 43 kDa | 0.15% |
| | OS = Bos taurus OX = 9913 | | | |
| | GN = CKM PE = 1 SV = 2 | | | |
| 15 | Cluster of Aldo-keto reductase family 1 | AKRIB1 | 36 kDa | 0.11% |
| | member B1 OS = Bos taurus | | | |
| | OX = 9913 GN = AKR1B1 | | | |
| | PE = 1 SV = 2 (P16116) | | | |
| 15.1 | Aldo-keto reductase family 1 member B1 | AKRIB1 | 36 kDa | 0.11% |
| | OS = Bos taurus OX = 9913 | | | |
| | GN = AKR1B1 PE = 1 SV = 2 | | | |
| 15.2 | Aldo_ket_red domain-containing protein | | 24 kDa | 0.08% |
| | OS = Bos taurus OX = 9913 | | | |
| | PE = 4 SV = 1 | | | |
| 16 | Acyl-CoA dehydrogenase long chain | ACADL | 48 kDa | 0.15% |
| | OS = Bos taurus OX = 9913 | | | |
| | GN = ACADL PE = 2 SV = 1 | | | |
| 17 | Aldo-keto reductase family 1 member Al | AKR1A1 | 37 kDa | 0.08% |
| | OS = Bos taurus OX = 9913 | | | |
| | GN = AKR1A1 PE = 2 SV = 1 | | | |
| 18 | 3-ketoacyl-CoA thiolase, mitochondrial | ACAA2 | 42 kDa | 0.08% |
| | OS = Bos taurus OX = 9913 | | | |
| | GN = ACAA2 PE = 1 SV = 1 | | | |
| 19 | Creatine kinase U-type, mitochondrial | CKMT1 | 47 kDa | 0.08% |
| | OS = Bos taurus OX = 9913 | | | |
| | GN = CKMT1 PE = 2 SV = 1 | | | |
| 20 | Citrate synthase, mitochondrial | CS | 52 kDa | 0.11% |
| | OS = Bos taurus OX = 9913 | | | |
| | GN = CS PE = 1 SV = 1 | | | |
| 21 | Cathepsin D OS = Bos taurus | CTSD | 42 kDa | 0.08% |
| | OX = 9913 GN = CTSD | | | |
| | PE = 3 SV = 1 | | | |
| 22 | Ribonuclease/angiogenin inhibitor 1 | RNH1 | 49 kDa | 0.08% |
| | OS = Bos taurus OX = 9913 | | | |
| | GN = RNH1 PE = 1 SV = 1 | | | |

Experiment 4. The identity of the active compound stimulating the growth of STEC and Salmonella was confirmed with commercially sourced biomolecules. The candidate proteins keratin and phosphoglucomutase were obtained and tested at two concentrations for growth stimulating activity to confirm the identity of the active compound (Table 9).

This demonstrated that the putative growth stimulating factor comprises phosphoglucomutase as suggested by the mass spectroscopy results of the excised active PAGE bands. The commercially sourced PGM demonstrated concentration dependent stimulation of E. coli O157:H7 growth as characterized in the crude F-1 and AS-20/60 fractions. Further, while the protein keratin was found to be abundant in the mass spectroscopy analysis, it clearly had no growth stimulating activity and demonstrated a mild inhibitory activity. See FIG. 1 depicting the experimental approach overview.

TABLE 9
Growth Stimulation of E. coli O157:H7^{a} by candidate
proteins obtained from commercial sources^{b}.

| | PGM (ug/mL) | | KRT (ug/mL) | |
|---|---|---|---|---|
| Control^{c} | 50 | 100 | 50 | 100 |
| 1.5 ± 0.10 | 2.1 ± 0.11^{e} | 2.4 ± 0.03^{e} | <LOD^{d} | 1.0 ± 0.12^{e} |

^{a}Values represent mean Log₁₀ ± standard deviation CFU/mL attained by a 1-3 CFU/mL of E. coli O157:H7 following 7 h incubation at 42° C.
^{b}Commercially available rabbit muscle phosphoglucomutase (PGM) and keratin (KRT) were supplemented at two concentrations into SSS media broth.
^{c}Control was non-supplemented SSS media broth.
^{d}Value below the level of detection (LOD) of 0.0 Log₁₀ CFU/mL.
^{e}The difference between the value for this this protein at this concentration compared to the control is significantly different (P < 0.05).

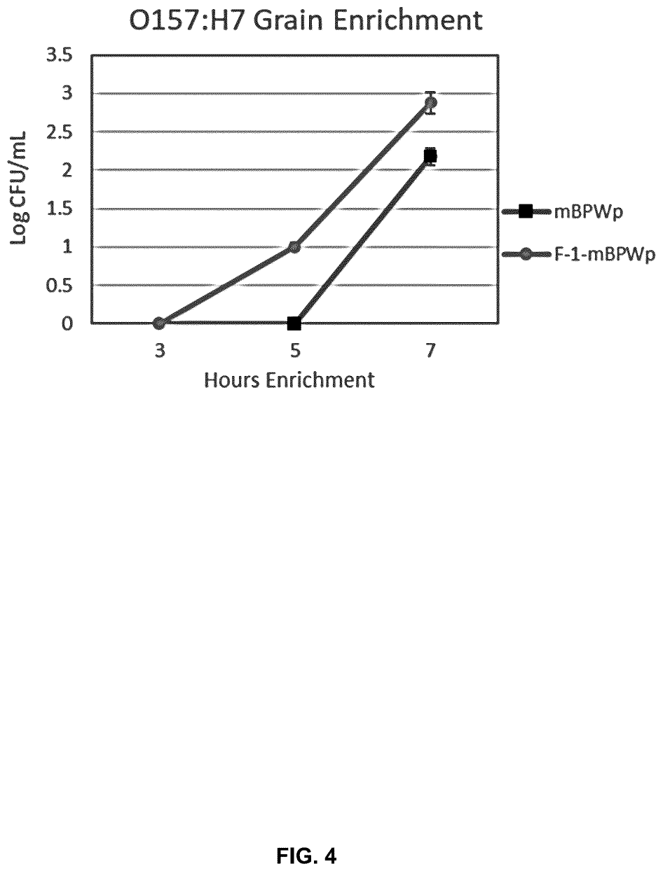

We anticipated that the use of PGM in an enrichment medium would decrease the time to detection for STEC and Salmonella, especially for samples that are not beef or meat. To examine this, an enrichment of wheat inoculated with E. coli O157:H7 was carried out in mBPWp and mBPWp supplemented with the PGM containing F-1 fraction. The growth curves from this demonstration showed that the PGM present in the F-1 fraction stimulated the growth of the E. coli O157:H7 over the control and would lead to more rapid detection (FIG. 4).

### Discussion

We entered into these experiments because we observed that inoculated spinach enrichments using the selective medium, SSS, at seven hours enrichment no detectable STEC colonies were found on plating medium while in comparable meat enrichments there were easily detectable numbers of STEC colonies. This suggested that there was either an active component provided by beef, or that there was an inhibitory compound supplied by the spinach. It could also be argued that since SSS media uses a number of components that inhibit background microflora growth, the beef was releasing a substance that negated the selectivity of the SSS media. However, since SSS media has been well characterized and defined for use in beef, and some control STEC and Salmonella strains grow slowly as a pure culture in SSS media [9], we decided to test the hypothesis that there was a component inherent in meat that was enhancing the growth of STEC and Salmonella.

We proceeded to isolate and characterize the growth stimulating activity extractable from beef tissues. Conditioned medium was shown to have 50-fold greater growth of E. coli O157:H7 than cultures of non-supplemented medium; and the same conditioned medium exhibited over 300-fold greater growth than the non-supplemented medium. The active component was shown to be precipitable in saturated ammonium sulfate solution permitting partial purification of the protein. Greater than 90% of the growth stimulating activity could be obtained by taking the 20%-60% ammonium sulfate saturation interval. As with the STEC growth stimulation effect, we demonstrated that the protein stimulates the growth of several Salmonella serotypes.

One of the earliest media used to cultivate bacteria was one that contained an infusion of meat [28]. Beef or meat extract has been a commonly used nutrient source in microbiology ever since. Current beef heart infusion (BHI) or beef extract powders are intended to replace the classical aqueous infusions of meat in culture media. Typical preparations of beef extract is a mixture of peptides, amino acids, nucleotides, organic acids, minerals and some vitamins. Manufacture of BHI and beef extract powders employ techniques that can hydrolyze or denature the activity of PGM when it is present. We suspect that this is why these media supplements lack the activity we identified in our experiments.

Fratamico et al [16] demonstrated that ground beef extracts activated genes associated with E. coli survival, particularly those associated with acid shock exposure. Although their findings did not hint at the apparent growth stimulating effects of a ground beef extractable protein. Harhay et al [17] found that ground beef enrichments supported more rapid growth of Salmonella than parallel control enrichments in mTSB. The authors were focused on the impact of this observation on the prediction model accuracy rather than theorize on the reasons for the reduction in doubling time for both the slow and fast-growing Salmonella strains in media containing ground beef versus only mTSB.

After obtaining a more highly purified preparation from affinity chromatography we noted that the predominant bands, 35 kD, 42 kD and 52 kD SDS PAGE bands were within the 50 kD-100 kD molecular weight range from the ultrafiltration experiments, assuming that the smaller proteins might exist as dimers. To verify that these proteins were growth stimulatory we isolated them from PAGE gels under minimally denaturing conditions. The assay for E. coli growth stimulating activity identified three PAGE bands, two of which were submitted for mass spectroscopy along with one inactive band to determine their identities. Common proteins were identified by the MS analysis in the active bands that were absent from the inactive band. Keratin was initially considered to be the candidate protein, however, it was abundant and appeared in both the active and inactive band MS analyses. The 42 kD band was rich in creatine phosphokinase which has been reported to be growth inhibitory towards E. coli [15]. The single protein found in the MS spectrum of both active bands (the 35 kD and 52 kD PAGE bands) analyzed was phosphoglucomutase (PGM). Purchased commercial rabbit muscle PGM was shown to exhibit appreciable E. coli growth stimulating activity while commercial keratin was devoid of growth stimulating activity.

The enzyme, PGM (E.C. 5.4.2.2), plays a central role in intermediary metabolism of glucose by inter-converting glucose1-phosphate with glucose-6-phosphate allowing the latter to enter the glycolytic pathway to generate cellular energy [20]. PGM mutants in E. coli are defective in their ability to utilize galactose as a carbon source since it cannot be converted to glucose 6-phosphate from glucose-1-phosphate, which ultimately generates energy via the glycolytic pathway [21]. Patterson et al. reported that PGM deletion mutants in Salmonella serotype Typhimurium were defective in O-antigen synthesis; were more susceptible to antimicrobial peptides and were less able to survive in infected mice than the wild-type strain [22]. The authors concluded that PGM played a critical role in imparting fitness and adaptability to Salmonella Typhimurium. These findings appear to comport with our observations that PGM stabilizes the growth of STEC and Salmonella in a highly selective medium.

STEC and Salmonella commandeer the catecholamines in the gastrointestinal tract to stimulate their growth through induction of an autoinducer molecule [23, 24]. An example of bacterial symbionts assimilating host enzymes to stimulate their growth is novel. Since the structure and function of PGM is evolutionarily conserved [25], it is possible that bacterial assimilation may be more readily achieved. The ability to commandeer host enzymes for survival and growth gives bacterial symbionts remarkable environmental adaptability. A recent in-silico study [26] examined numerous host pathogen protein interactions and implicated several bacterial enzymes and proteins in the pathogenesis process, however nothing quite like the assimilation of particular host proteins to facilitate pathogen adaptation and survival in the host environment.

### CONCLUSION

In conclusion, we recognized and identified PGM as a growth stimulating substance in ground beef extracts. While much of this study was conducted utilizing bovine tissues, PGM is present at varying levels in all eukaryotic organisms. Its greater activity in meat samples compared to spinach may be due to the cell wall of plants prohibiting its release into the enrichment medium. Although these results need further development current data indicate that PGM can serve as a supplement in numerous enrichment media to improve pathogen detection.

### Example 2. Preparation of Yeast PGM, a Potent Gram-Negative Microbial Growth Stimulant

From the studies conducted to date we have demonstrated that phosphoglucomutase is a growth stimulating substance. While the original studies were conducted using ground beef and ground rumen, the use of these materials as potential sources of PGM have process drawbacks. Notably the fatty nature of bovine tissues results in crude and filtered extracts which are high in lipids and lipoproteins which tend to coat microporous filtration membranes, fouling them and rendering them unusable for filter sterilization operations. We have found that baker's yeast is a rich source of phosphoglucomutase but not having all the fat associated with animal tissues. The following example provides details on the preparation of partially purified yeast PGM suitable for scale production of this valuable protein.

One kilogram of Fleischmann's baker's yeast was purchased from Amazon. We used a modification of the purification procedure described by Daugherty et al [33]. Two hundred grams of dry yeast was dispersed with stirring in 500 mL of 0.07M Na₂1HPO₄ containing 0.0001M EDTA, 0.5 mL toluene at 38° C. for four hours. The suspension was filtered through fast flow filter paper to remove the autolyzed yeast cake. The residual yeast cake was re-suspended in 250 mL of phosphate buffer and stirred for 30 minutes and re-filtered through a paper filter. The supernatants were combined and filtered through a glass fiber filter to remove any residual fine particles. The resulting supernatant was filter sterilized by filtration through a sterile 0.22 μm pore size polyether sulfone membrane filtration unit obtained from Millipore.

The sterile filtrate was assayed for STEC growth stimulating activity by inclusion in 3.0 mL of mSTEC medium with or without 10% v/v of the sterile supplement. Partially purified bovine liver PGM was included in the assay for comparison of the relative activity of the two protein preparations. Table 10 depicts the plate count data for a seven-hour enrichment.

TABLE 10
Effect of yeast PGM on STEC growth

| Sample | 7-h Plate CFU/mL |
|---|---|
| Control (- PGM) | 20 |
| 10% Bovine Liver PGM | 1850 |
| 10% Yeast PGM | TNTC (>80,000 ) |

The actual plate counts were readily done with the control and bovine liver samples, while the yeast PGM containing sample yielded a continuous lawn of mauve indistinguishable colonies indicating that were more than 800-900 colonies on the plate. By our estimate, the yeast PGM preparation has 5 to 10-fold more growth stimulating activity than the bovine liver preparation. The advantages of using yeast as a source of PGM are manifold over animal tissue sources: 1) Easily accessible and stored at room temperature; 2) Purification of the active PGM is substantially easier and less cost; 3) Preparations of the yeast enzyme appear to have higher growth stimulating activity than the bovine liver enzyme.

### Example 3. Extraction from the Growth Stimulating Factor from Equine Source

1.5 g horse liver acetone powder (L9627, Sigma-Aldrich, St. Louis, MO) was incubated in 15 mL of 0.064 M magnesium chloride and 0.05% v/v Niaproof-4 in water. The resulting liquid was filtered through a glass fiber filter and filter sterilized with a 0.2 μm filter to generate a horse liver acetone powder extract.

One mL of mBPWp with and without supplementation with the horse liver acetone powder extract at a final concentration of 10% v/v was inoculated with ˜10 CFU/mL at time zero and incubated at 37° C. for six hours. One hundred microliters of each culture was plated onto Chromagar STEC and incubated 18 hours at 37° C. Mauve colonies were enumerated the following morning. Table 11 depicts the results.

TABLE 11

| Sample | CFU/mL |
|---|---|
| Control | 200 |
| Horse Liver Acetone Powder Extract | >12500 |

This example shows that the growth stimulating factor described herein can be extracted from equine tissues as well as leporine and bovine tissues.

### Example 4. Salmonella study in Ground Turkey

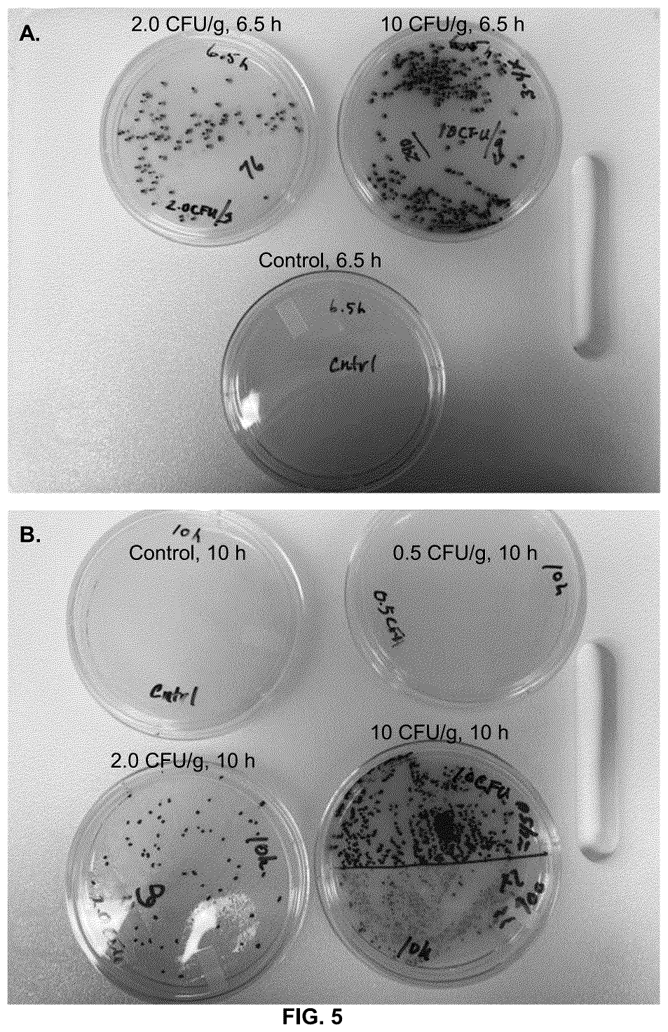

25 g ground turkey was inoculated at 0.5, 2.0, 10.0 CFU/g with S. Typhimurium. After incubating the inoculated turkey for twenty minutes at room temperature, PDX-STEC medium supplemented with a growth stimulating factor preparation (i.e., the 20-60% ammonium sulfate cut of ground beef extract from Example 5) to a final concentration of 10% v/v was added to the inoculated turkey, and the inoculated was incubated a further 6.5 hours and observed for color transition. A further time point was observed at 10 hours. Neither the 6.5-hr nor the 10-hr samples were yellow in color. 50 microliters of the 10-hr enrichments were plated onto Chromagar Salmonella Plus. FIG. 5 shows the marked difference in the 10 CFU/g sample versus the control, 0.5 CFU/g, and 2.0 CFU/g samples at 6.5 hr enrichment and 10 hr enrichment.

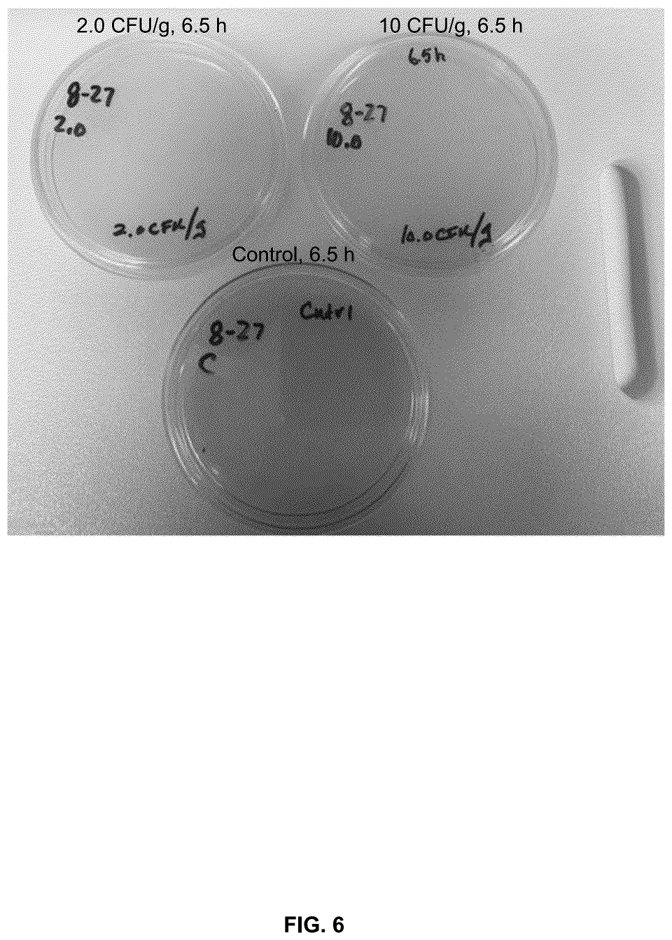

By contrast, 25 g turkey samples, including a negative control, 2.0 CFU/g, and 10.0 CFU/g enriched in PDX-STEC medium without supplementation with the growth stimulating factor preparation were all negative in platings done at 6.5 h enrichment. See FIG. 6. All samples were yellow in color after 18 hour enrichment at 40° C.

Plate counts of the enrichments done in medium containing the growth stimulating factor preparation could be shown to exhibit a roughly 3-4 fold increase in Salmonella population between the 2.0 CFU/g initial inoculation and the 10.0 CFU/g inoculation levels. Samples plated from the ten-hour enrichments demonstrated a larger population count difference of roughly ten-fold difference.

A summary of the findings from the present example is shown in Table 12.

TABLE 12
Summary of findings from the present example.

| | CFU/mL − | CFU/mL + |
|---|---|---|
| Sample | supplement | supplement |
| Cntrl-6.5 h | 0 | 0 |
| 2.0 CFU/g-6.5 h | 0 | 760 |
| 10.0 CFU/g-6.5 h | 0 | 2900 |
| Cntrl-10 h | 0 | 0 |
| 2.0 CFU/g-10 h | 0 | 700 |
| 10 CFU/g-10 h | 0 | 9000 |

These results show that supplementation of growth medium with the growth stimulating factor significantly reduced the time to detection of positive samples compared to negative controls and was capable of detecting low-level S. Typhimurium inoculations of raw ground turkey. These results also show that use of the growth stimulating factor is capable of quantitatively distinguishing samples with low levels of contamination from those having high-level contamination at an early stage of sample enrichment, especially when coupled with qPCR analysis for the target pathogen.

### REFERENCES

1. Mckay, B. Aurora Packing Company, Inc. Recalls Beef Products Due to Possible E. coli O157:H7 Contamination. Available at www.fsis.usda.gov/recalls-alerts/aurora-packing-company-inc.-recalls-beef-products-due-possible-e.-coli-o157h7.
2. Okonta, C. FSIS Issues Public Health Alert for Raw Beef Ravioli Products Due to Possible E. Coli O157:H7 Contamination. Available at www.fsis.usda.gov/recalls-alerts/fsis-issues-public-health-alert-raw-beef-ravioli-products-due-possible-e.-coli.
3. Hirschmugl, J. Meijer Recalls Whole Cantaloupes and Select Cut Cantaloupe Trays Due to Potential Health Risk. Available at www.fda.gov/safety/recalls-market-withdrawals-safety-alerts/meijer-recalls-whole-cantaloupes-and-select-cut-cantaloupe-trays-due-potential-health-risk.
4. Seneca Recalls Cinnamon Apple Chips Because of Possible Health Risk. Available at www.fda.gov/safety/recalls-market-withdrawals-safety-alerts/seneca-recalls-cinnamon-apple-chips-because-possible-health-risk.
5. Prima® Wawona Recalls Bulk/Loose and Bagged Peaches Due to Possible Salmonella Risk. Available at www.fda.gov/safety/recalls-market-withdrawals-safety-alerts/primar-wawona-recalls-bulkloose-and-bagged-peaches-due-possible-salmonella-risk.
6. King Arthur Flour Updates Three Lot Codes of Voluntarily Recalled Unbleached All-Purpose Flour (5 lb.) Available at www.fda.gov/safety/recalls-market-withdrawals-safety-alerts/king-arthur-flour-updates-three-lot-codes-voluntarily-recalled-unbleached-all-purpose-flour-5-lb.
7. Ceuppens, S., Li, D.; Uyttendaele, M.; Renault, P.; Ross, P.; Van Ranst, M.; Cocolin, L. and Donaghy, J. (2014). Molecular Methods in Food Safety Microbiology: Interpretation and Implications of Nucleic Acid Detection. Compr. Rev. Food Sci. Food Saf., 13(4), 551-577.
8. Sunar, N. M., Stentiford, E. I., Stewart, D. I., and Fletcher, L. A. (2010). Molecular techniques to characterize the invA genes of Salmonella spp. for pathogen inactivation study in composting. Available at: arxiv.org/abs/1404.5208.
9. Eggers, J., Feirtag, J. M., Olstein, A. D., and Bosilevac, J. M. (2018). A Novel Selective Medium for Simultaneous Enrichment of Shiga Toxin-Producing Escherichia coli and Salmonella in Ground Beef. J Food Prot., 81(8), 1252-1257.
10. U.S. Department of Agriculture, Food Safety Inspection Service. (2014). Microbiology laboratory guidebook. Method no. 5B.05.
11. Olstein, A. D. (2016). Selective enrichment media and uses thereof. U.S. Pat. No. 9,518,283.
12. Turner, A. J. (1979). A Simple and Colorful Procedure to Demonstrate the Principles of Affinity Chromatography. Biochem. Ed., 7(3), 60-62.
13. Leammli, U. K. (1970). Cleavage of structural proteins during the assembly of the head of bacteriophage T4. Nature, 227(5259), 680-5.
14. Simpson, R. J. (2007). Zinc/Imidazole Procedure for Visualization of Proteins in Gels by Negative Staining. CSH Protoc., pdb.prot4701.
15. An, F., Fan, N. and Zhang, S. (2009). Creatine kinase is a bacteriostatic factor with a lectin-like activity. Mol. Immun., 46 (13), 2666-2670.
16. Fratamico P. M., Wang S., Yan X., Zhang W., Li Y. (2011). Differential gene expression of E. coli O157:H7 in ground beef extract compared to tryptic soy broth. J Food Sci., 76(1), M79-87.
17. Harhay, D. M., Weinroth, M. D., Bono, J. L., Harhay, G. P., and Bosilevac, J. M. (2020). Rapid estimation of Salmonella enterica contamination level in ground beef—Application of the time-to-positivity method using a combination of molecular detection and direct plating. Food Microb., 93, 103615-103623.
18. Turro, N. J., Lei, X., Ananthapadmanabhan, K. P., and Aronson, M. (1995). Spectroscopic probe analysis of protein-surfactant interactions: The BSA/SDS system. Langmuir, 11, 2525-2533.
19. Thu, Y. M., Van Riper, S. K., Higgins, L., Zhang, T., Becker, J. R., Markowski, T. W., Nguyen, H. D., Griffin, T. J., Bielinsky, A. K. (2016). Slx5/Slx8 Promotes Replication Stress Tolerance by Facilitating Mitotic Progression. Cell Rep., 15(6), 1254-65.
20. P.540, Chap. 9 in Biochemistry, The Chemical Reaction of Living Cells. David Metzler ed. Academic Press. 1977.
21. Adhya, S. & Schwartz, M. (1971). Phosphoglucomutase mutants of Escherichia coli K-12. J Bacteriology, 108, 621-626.
22. Patterson, G. K., Cone, D. B., Peters, S. E. and Maskell, D. J. (2009). The enzyme phosphoglucomutase (PGM) is required by Salmonella enterica serovar Typhimurium for O-antigen production, resistance to antimicrobial peptides and in vivo fitness.

Microbiology, 155, 3403-3410.
23. Lyte, M., Frank, C. D., and Green, B. T. (1996). Production of an autoinducer of growth by norepinephrine cultured Escherichia coli O157:H7. FEMS Microbiol. Lett., 139, 155-159.
24. Pullinger, G. D., Carnell, S. C., Sharaff, F. F., van Dieman, P. M., Dziva, F., Morgan, E., Lyte, M., Freestone, P. P. E. and Stevens, M. P. (2010). Norepinephrine Augments Salmonella enterica-Induced Enteritis in a Manner Associated with Increased Net Replication but Independent of the Putative Adrenergic Sensor Kinases QseC and QseE. Infect. Immun., 78(1), 372-380.
25. Mehra-Chaudhary, R., Mick, J., Tanner, J. J., Henzle, M. T. and Beamer, L. J. (2011). Crystal structure of a bacterial phosphoglucomutase, an enzyme involved in the virulence of multiple human pathogens. Proteins, 79(4), 1215-1229.
26. Bose, T., Venkatesh, K. V. and Mande, S. S. (2019). Investigating host-bacterial interactions among enteric pathogens. BMC Genom., 20, 1022.
27. U.S. Food and Drug Administration. Peter Feng and Karen Jinneman B A M: Diarrheagenic Escherichia coli. 2017. Available at www.fda.gov/food/laboratory-methods-food/bam-chapter-4a-diarrheagenic-escherichia-coli.
28. Huntoon, F. M. (1918). “Hormone” Medium: A Simple Medium Employable as a Substitute for Serum Medium, J. Infect. Dis., 23(2), 169-172.
29. Snyder, T. R., Boktor, S. W., & M'ikanatha, N. M. (2019). Salmonellosis Outbreaks by Food Vehicle, Serotype, Season, and Geographical Location, United States, 1998 to 2015. J. Food Prot., 82(7), 1191-1199.
30. Brooks, J. T., Sowers, E. G., Wells, J. G., Greene, K. D., Griffin, P. M., Hoekstra, R. M., & Strockbine, N. A. (2005). Non-0157 Shiga toxin-producing Escherichia coli infections in the United States, 1983-2002. J. Infect. Dis., 192(8), 1422-1429.
31. Centers for Disease Control and Prevention (CDC). (2007). Multistate outbreak of Salmonella serotype Tennessee infections associated with peanut butter—United States, 2006-2007. MMWR Morb Mortal Wkly Rep., 56(21), 521-524.
32. Bosilevac, J. M., Kalchayanand, N., Schmidt, J. W., Shackelford, S. D., Wheeler, T. L., and Koohmaraie, M. (2010). Inoculation of beef with low concentrations of Escherichia coli O157:H7 and examination of factors that interfere with its detection by culture isolation and rapid methods. J Food Prot., 73(12), 2180-8.
33. Daugherty, J. P., Kraemer, W. F. and Joshi, J. F. (1975). Purification and Properties of Phosphoglucomutase from Fleischmann's Yeast. Eur. J. Biochem., 57, 115-126.

1. A microbial growth medium, comprising:
a growth-stimulating amount of phosphoglucomutase; and
a component comprising a carbon and nitrogen source, a fermentable sugar, an inorganic salt, or any combination thereof.

2. The microbial growth medium of claim 1, wherein the microbial growth medium is sterile.

3. The microbial growth medium of claim 1, wherein the microbial growth medium comprises an efflux pump inhibitor.

4. The microbial growth medium of claim 3, wherein the efflux pump inhibitor comprises one or more of a phenothiazine, a phenylpiperidine, a tetracycline analog, an aminoglycoside analog, a fluoroquinolone analog, a quinoline derivative, a peptidomimetic, a pyridopyrimidine, an arylpiperidine, and an arylpiperazine.

5. The microbial growth medium of claim 3, wherein the efflux pump inhibitor comprises one or more of an arylpiperazine and a quinoline derivative.

6. The microbial growth medium of claim 3, wherein the efflux pump inhibitor comprises one or more of 1-(1-naphthylmethyl)piperazine and 4-chloroquinoline.

7. The microbial growth medium of claim 1, wherein the microbial growth medium comprises a selective agent comprising one or more of a sulfa drug, a surfactant, an aminocoumarin, cycloheximide, myricetin, nitrofurantoin, a rifamycin, a polyketide, and an oxazolidinone.

8. The microbial growth medium of claim 7, wherein the sulfa drug comprises one or more of sulfanilamide and sulfathiazole, the surfactant comprises 7-ethyl-2-methyl-4-undecyl sulfate or a salt thereof, the aminocoumarin comprises novobiocin, the rifamycin comprises rifampicin, the polyketide comprises doxycycline, and the oxazolidinone comprises linezolid.

9. The microbial growth medium of claim 7, wherein the selective agent further comprises one or more of a supravital stain, ascorbic acid, and bromobenzoic acid, wherein the fermentable sugar comprises 2-deoxy-D-Ribose and the supravital stain comprises brilliant green.

10. The microbial growth medium of claim 7, wherein the selective agent and the efflux pump inhibitor are present in amounts effective to inhibit growth of at least one non-Salmonella species to a greater extent than one or more Salmonella species.

11. The microbial growth medium of claim 7, wherein the selective agent and the efflux pump inhibitor are present in amounts effective to inhibit growth of at least one non-Shiga toxin-producing E. coli strain to a greater extent than one or more Shiga toxin-producing E. coli strains.

12. The microbial growth medium of claim 1, further comprising a visual indicator that indicates the presence of a microorganism selected from the group consisting of Salmonella and E. coli.

13. The microbial growth medium of claim 1, wherein the microbial growth medium is devoid of lipid or contains lipid in an amount less than 5% w/w.

14. The microbial growth medium of claim 1, wherein the phosphoglucomutase comprises a phosphoglucomutase other than bovine phosphoglucomutase.

15. The microbial growth medium of claim 1, wherein the phosphoglucomutase comprises yeast phosphoglucomutase.

16. The microbial growth medium of claim 1, wherein the phosphoglucomutase is provided in the form of a partially purified yeast protein preparation.

17. A method of growing a microorganism, comprising culturing a sample suspected of containing the microorganism in the microbial growth medium of claim 1.

18-23. (canceled)

24. A method of growing a microorganism, comprising culturing a sample suspected of containing the microorganism in a microbial growth medium comprising a growth-stimulating amount of phosphoglucomutase.

25-32. (canceled)

33. The method of claim 24, wherein the phosphoglucomutase comprises a phosphoglucomutase other than bovine phosphoglucomutase.

34. The method of claim 24, wherein the phosphoglucomutase comprises yeast phosphoglucomutase.

35-53. (canceled)
